(19) United States
(12) Patent Application Publication
(10) Pub. No.: US 2020/0365258 A1
(43) Pub. Date: Nov. 19, 2020

(54) APPARATUS FOR GENERATING AND TRANSMITTING ANNOTATED VIDEO SEQUENCES IN RESPONSE TO MANUAL AND IMAGE INPUT DEVICES

(71) Applicants: DAVID J. LANGER, NEW YORK, NY (US); GREGORY ODLAND, MOUNT KISCO, NY (US); KENNETH H. COURT, BROOKLYN, NY (US)

(72) Inventors: DAVID J. LANGER, NEW YORK, NY (US); GREGORY ODLAND, MOUNT KISCO, NY (US); KENNETH H. COURT, BROOKLYN, NY (US)

(21) Appl. No.: 16/876,083

(22) Filed: May 17, 2020

Related U.S. Application Data
(60) Provisional application No. 62/849,716, filed on May 17, 2019.
(60) Provisional application No. 62/947,544, filed on Dec. 13, 2019.

(51) Int. Cl. G16H 40/20; G11B 27/036; G06F 3/0488; H04N 5/93; G16H 30/20; G16H 30/40; G06Q 10/06; G16H 40/63; G16H 20/10; G16H 20/30; G16H 50/50; G06Q 10/10; G06F 16/71; G06F 16/74; G06F 16/735; G09B 5/06; G09B 19/00

(52) U.S. Cl. CPC G16H 40/20; G16H 80/00; G06F 3/04883; H04N 5/9305; G16H 30/20; G16H 30/40; G06Q 10/06316; G16H 40/63; G16H 20/10; G16H 20/30; G16H 50/50; G06Q 10/107; G06Q 10/1097; G06F 16/71; G06F 16/74; G06F 16/735; G09B 5/065; G09B 19/003; G11B 27/036

(57) ABSTRACT

In accordance with the invention, a health care information generation and communication system comprises a body part image generation device for generating body part image information representing a body part of a patient. A body part image database is coupled to receive the output of the body part image generation device and store the image information as a stored image. A stored image playback device is coupled to the body part image database and generating a recovered image from the image information. An image control device is coupled to the stored image playback device to select a desired portion of the body part image information and output the selected portion as a selected image. A video generation device is coupled to the image control device to receive the selected image from the stored image playback device and coupled to a microphone and combine the same into an output video. The output video thus comprises visual and audible elements. A video database is coupled to receive the visual and audible elements of the output video from the output of the video generation device and store the visual and audible elements. A video player presents a display of at least a portion of the visible and audible elements.

### CROSS REFERENCE TO RELATED APPLICATIONS

This application claims the priority of the U.S. Provisional Patent Application No. 62/849,716 filed May 17, 2019 and entitled Patient Communication and Notification System with Provider and Patient Multiply Sourced and Updated Databases and Information Communications Backbone, and also claims the priority of U.S. Provisional Patent Application No. 62/947,544 filed Dec. 13, 2019 and entitled Healthcare Provider and Patient Communications System. The disclosures of both of the above provisional patent applications are hereby incorporated herein by reference.

### TECHNICAL FIELD

The invention relates to apparatus and methods for receiving and integrating anatomical images, manual and/or audio inputs, for example from healthcare providers, and transmitting the same to other care providers, the patient and optionally to family members and/or other nonprofessional persons associated with the patient.

### BACKGROUND OF THE INVENTION

In the normal course of medical treatment, a patient will provide information such as name, address, allergies, medications, symptoms and so forth to a healthcare provider. This information is recorded in a patient medical record. During the course of treatment, the patient record is supplemented by such things as test results, drug and treatment information and medical imaging.

Critical to the success of the treatment, the patient is, after examination, given instructions and/or medications which may constitute the totality of the medical treatment. Alternatively, the medical treatment may also involve a surgical procedure, other treatments, such as dialysis, and so forth.

Generally, whether the treatment is to constitute medications, exercise, treatments such as dialysis, or surgery, the doctor communicates the patient's condition using images such as x-rays, cautions the patient respecting side effects or other potential artifacts of treatment, and communicates to the patient information respecting what the patient should be doing and looking out for. Most often, this information is provided orally. Sometimes, the oral information may be supplemented by a written set of instructions, such as instructions about fasting before a colonoscopy.

### SUMMARY OF THE INVENTION

The above procedures, which, by and large, reflect current medical practice, while highly effective in advancing medical objectives, also suffer from the disadvantage of being reliant upon both patient understanding and to a large extent patient memory for their effectiveness. However, experience has shown that this reliance, while well-placed, can result in a situation where information is forgotten or misunderstood. While written instructions help to some extent, patients are unlikely to carry such instructions around with them, and thus likely to neglect the tasks which they are being relied upon to complete.

In addition, the success of medical treatment also often relies on the cooperation and health of persons around the patient, such as their family. However, the above procedures often result in a substantial information gap between the medical provider and the family of the patient. Indeed, in the context of perfect communication between patient and family, the best case scenario possible is for the family to have the same understanding of the medical condition and what needs to be done as the patient. However, as a practical matter, there is a high likelihood that the imperfect knowledge base acquired by the patient during treatment will be only partially communicated to the family, and further that such patient to family communication will include errors.

In accordance with the invention, a method and apparatus for enhancing compliance with patient instructions is provided.

More particularly, in accordance with one embodiment of the invention, information contained in the patient record is presented to the patient electronically over a publicly accessible network.

In accordance with the inventive system, it is contemplated that a video containing information respecting the treatment of the patient is created during the course of medical treatment and made accessible over the publicly accessible network.

Yet further in accordance with the invention, any supplemental information given to the patient, for example by telephone, may be added to the patient record and/or the video and thus be accessible to the patient at a future date to ensure that communication has been thorough and that there are no questions left unanswered. However, immutable earlier records are archived for record-keeping and evidentiary purposes, including protection against legal claims.

In accordance with the invention, the doctor, nurse or other clinician pulls out the most important information, images, and so forth and puts together the elements which will eventually be, for example, a video which becomes a remotely accessible patient record, which may optionally be immutable, or be unalterably stored in its original and subsequent forms. These elements may include various medical records, x-ray images, drug identity, etc.

In accordance with the invention, a health care information generation and communication system comprises a body part image generation device for generating body part image information representing a body part of a patient. A body part image database is coupled to receive the output of the body part image generation device and store the image information as a stored image. A stored image playback device is coupled to the body part image database and generates a recovered image from the image information. An image control device is coupled to the stored image playback device to select a desired portion of the body part image information and output the selected portion as a selected image. A video generation device is coupled to the image control device to receive the selected image from the stored image playback device. The video generation device is coupled to a microphone and combines the output of the same into an output video. The output video thus comprises visual and audible elements. A video database is coupled to receive the visual and audible elements of the output video from the output of the video generation device and store the visual and audible elements. A video player presents a display of at least a portion of the visual and audible elements.

The body part image information may be displayed as i) a plurality of two dimensional images representing different body parts, ii) views with different magnifications of one or more body parts, iii) different views of one or more body parts, or iv) partial views of one or more body parts.

The body part image information may be selected from the group consisting of i) still images, ii) moving images, iii) x-ray images, iv) ultrasound images, v) optical images, vi) MRI images, and vii) other medical images. The recovered image may be a two-dimensional image.

The input device may be selected from the group consisting of a tablet, a touchscreen and an alpha numeric generating device.

A video display device may be used to display the output video as it is generated in real time. Touchscreen elements may be associated with the video display device or a tablet. The touchscreen elements or tablet may be configured to receive a manual input, such as a circle encircling a part of an image displayed on the video display device from a person operating the video generation device. An alpha numeric generating device, such as a keyboard, may be coupled to input alphanumeric information in the video generation device to implement display of the alphanumeric information in the output video.

The video generation device may comprise a non-volatile storage medium having stored thereon a template for the output video, the template presenting directions to the person operating the video generation device and presenting screens for the entry of alphanumeric information to be incorporated into the output video.

The system may further comprise alphanumeric data generating healthcare instrumentation. Such instrumentation generates alphanumeric data. The alphanumeric data generating healthcare instrumentation is coupled to the video generation device. The video generation device may be responsive to a control signal input by a person operating the video generation device to incorporate at least a portion of the alphanumeric data into the output video.

In accordance with the invention, it is contemplated that a video and patient record database may be divided into a plurality of patient sectors (for example on a hard drive, or non-volatile memory device), with, for example, each of the patient sectors associated with an individual patient. The video database is coupled to receive the visual and audible elements of the output video from the output of the video generation device and store the visual and audible elements in a patient sector associated with the particular individual patient. Advantageously, a publically accessible network to which a server is linked may make information in the video database and the other databases available over the publically accessible network, for example to medical professionals and patient smartphones associated with the particular individual patient. In accordance with the invention, the smartphones have downloaded thereon an application for accessing and providing patient specific identification information and accessing the server over the publically accessible network to cause the server to access the video and other databases and transmit the contents of the same, for example, a video associated with the particular individual patient, to the patient smartphone or the smartphones of providers, allowing repeated study of the same, whenever the patient, healthcare professional or other associated individual desires to access the same.

The inventive system may also further comprise an input device selected from the group consisting of a tablet, a touchscreen and an alpha numeric generating device.

In accordance with the invention it is also contemplated that a video display device may be provided for displaying the output video as it is generated in real time;

Touchscreen elements associated with a video display device or a tablet, may be used to receive a manual input, such as a circle encircling a part of an image displayed on the video display device from a person operating the video generation device. An alpha numeric generating device is coupled to input alphanumeric information into the video generation device to implement display of the alphanumeric information in the output video.

The video generation device may comprise a non-volatile storage medium having stored thereon a template for the output video, the template presenting directions to the person operating the video generation device and presenting screens for the entry of alphanumeric information to be incorporated into the output video.

Alphanumeric data generating healthcare instrumentation generating alphanumeric data may be employed to generate healthcare information and maybe coupled to the video generation device. The video generation device may be responsive to a control signal input by a person operating the video generation device to incorporate at least a portion of the alphanumeric data into the output video.

The platform provided by the inventive system also contemplates optionally presenting screens to the patient for enabling the patient to access a healthcare provider or other person associated with the medical treatment of the patient by way of email and/or telephone.

In accordance with the invention, an image of a treatment protocol prescription, such as pre-op directions, wound care directions, medication directions, post-op directions, physical therapy and/or exercise directions or the like, may be created. An image of a part of the body related to a physiological issue, such as the lung, or ear or of a physiological parameter such as pressure or damage such as that produced by an x ray or MRI machine may be included in the video.

The video may be created by inputting a still and/or video image into a video recording system while creating an audiovisual sequence. In addition, an audio signal may be generated from the voice of a healthcare provider, and input into the video recording system while the inputting of a still and/or video image into a video recording system is in progress, to incorporate the audio signal into the audiovisual sequence to make the video. In addition, simultaneously, a pen and tablet input may also be incorporated into the video to input manually generated image elements into the audiovisual sequence, for example the circling of a physiological phenomenon or element which a doctor is speaking about. The video may then be made available over a network accessible to the patient.

In accordance with the invention, the patient record may comprise background information on the patient, such as medications, allergies, symptoms, medical history and the like.

As alluded to above, the inputting of the still and/or video image and the audio signal may be performed during the time that the patient is listening to and/or discussing their condition with their doctor.

The patient record may include each of a plurality of tasks which the patient is responsible for and times for performance of the same. Infrastructure is provided for notifying the patient at the appointed time, for example by the patient being emailed with a reminder to perform the particular task, and given the opportunity to confirm that the same has been done, and upon the failure to receive such a confirmation, a family member or member of the professional team may be notified that the task is not yet performed.

The inventive method also contemplates that the patient record may be archived in a form which may not be altered in order to serve as a permanent record to guide future actions.

Optionally, and in addition to the above, the databases associated with the inventive system may include sectors to receive data elements of the type associated with so-called “meaningful use” standards associated with effective care delivery in legislative, insurance, industry norm and other accepted protocols. These may include the use of datasets generated in accordance with the inventive system which are useful in complying with reporting of the type necessary to satisfy government requirements and/or federal reimbursements standards and/or insurance coverage. As insurance/reimbursement and related models migrate toward quality of care measurement by monitoring patient treatment elements and outcomes, the data sets maintained in the inventive system, including patient histories embodying such parameters as amount and nature of medications, duration of treatment, involvement and extent of involvement of healthcare providers and the amount of time that they spend, and so forth, may all be used to measure the quality of care.

In addition, the inventive integration, assembly and automated (optionally following, or partially or substantially independent of, human input) graphic layout of graphic, alphanumeric, audible and other inputs using manual, optical, alphanumeric (including alphanumeric information input by the healthcare provider or gathered by the system from public domain sources (optionally healthcare system reviewed information)) and other input devices results in a communications function which will improve patient outcomes. Moreover, all of this data can be generated by the system and may be used to comply with entitlement requirements, for example, a greater proportion of shared savings in accordance with various governmental and other programs, as well as to resolve any disputes.

### BRIEF DESCRIPTION OF THE DRAWINGS

The operation of the inventive infrastructure and method will become apparent from the following description taken in conjunction with the drawings, in which:

FIG. 1 is a block diagram generally illustrating a general implementation of the system of the present invention;

FIG. 2 is a block diagram illustrating an exemplary embodiment of a method in accordance with the present invention;

FIG. 3 is a block diagram generally illustrating an exemplary embodiment of the method of the present invention illustrating the same in the context of the discharge of a patient after surgery;

FIG. 4 is a block diagram illustrating an exemplary embodiment of a mobile app as implemented according to the present invention;

FIG. 5 illustrates a home screen in the mobile app of FIG. 4, in an exemplary implementation of the present invention;

FIG. 6 illustrates a gallery screen which enables access to images related to the treatment of a patient in the mobile app of FIG. 4, in an exemplary implementation of the present invention;

FIG. 7 illustrates the second page in the gallery screen of FIG. 6;

FIG. 8 illustrates a screen in the gallery of FIG. 6 which enables access to videos in an exemplary implementation of the present invention;

FIG. 9 illustrates a screen in the gallery of FIG. 6 which enables access to documents, in an exemplary implementation of the present invention;

FIG. 10 illustrates a screen in the gallery of FIG. 6 which enables access to audio records, in an exemplary implementation of the present invention;

FIG. 11 illustrates a screen in the mobile app of FIG. 4 which enables access to subcategories of information in an office visit category, in an exemplary implementation of the present invention;

FIG. 12 illustrates a screen which branches off the screen of FIG. 11 and which enables access to information about a patient's office visit, in an exemplary implementation of the present invention;

FIG. 13 illustrates a screen which enables access to information about the patient's office care team, in an exemplary implementation of the present invention;

FIG. 14 illustrates a screen which provides access to additional information, in an exemplary implementation of the present invention;

FIG. 15 illustrates a screen which provides access to information about pre-operation preparation, in an exemplary implementation of the present invention;

FIG. 16 illustrates a screen in the mobile app of FIG. 4 enabling access to information related to a hospital visit by a patient, in an exemplary implementation of the present invention;

FIG. 17 illustrates a screen accessed through FIG. 16 which provides access to the patient's discharge instructions, in an exemplary implementation of the present invention;

FIG. 18 illustrates a screen providing access to information about a patient's hospitalization, in an exemplary implementation of the present invention;

FIG. 19 illustrates a screen providing access to daily updates after the “Daily Updates” icon has been touched, in an exemplary implementation of the present invention;

FIG. 20 illustrates a screen accessed through the screen of FIG. 16 providing access to information related to the patient's medications, in an exemplary implementation of the present invention;

FIG. 21 illustrates a screen which provides access to information related to activities and restrictions, in an exemplary implementation of the present invention;

FIG. 22 illustrates a screen which provides access to information related to symptom management, in an exemplary implementation of the present invention;

FIG. 23 illustrates a screen which provides access to information such as a patient's wound care instructions, in an exemplary implementation of the present invention;

FIG. 24 illustrates a screen which provides access to information related to the patient's nutrition and diet, in an exemplary implementation of the present invention;

FIG. 25 illustrates a screen which provides access to information related to the hospital care team, in an exemplary implementation of the present invention;

FIG. 26 illustrates a screen in the mobile app of FIG. 4 which provides access to information related to the patient's responsibilities, in an exemplary implementation of the present invention;

FIG. 27 illustrates a screen in the mobile app of FIG. 4 which provides access to information about the internal professional care team helping the patient, in an exemplary implementation of the present invention;

FIG. 28 illustrates a screen which provides access to information about the external care team of the hospital, in an exemplary implementation of the present invention;

FIG. 29 illustrates a screen which provides access to information about a personal care team, such as support of family members, in an exemplary implementation of the present invention;

FIG. 30 illustrates a screen in the mobile app of FIG. 4 which displays information relating to and provides access to notifications, in an exemplary implementation of the present invention;

FIG. 31 is a block diagram illustrating details of an exemplary embodiment of a mobile app according to the present invention;

FIG. 32 illustrates a screen in the mobile app of FIG. 31 which displays information relating to and provides access to a share tool, in an exemplary implementation of the present invention; and

FIG. 33 illustrates a screen in the mobile app of FIG. 31 which displays information relating to and provides access to an invite tool, in an exemplary implementation of the present invention.

### DETAILED DESCRIPTION OF THE PREFERRED EMBODIMENTS

In accordance with the invention, reliable communication between the clinician and the clinician's team on the one hand and the patient and the patient's family or other lay support group is ensured while providing both the patient team and the clinician team access to information and real-time communication with all persons in all groups.

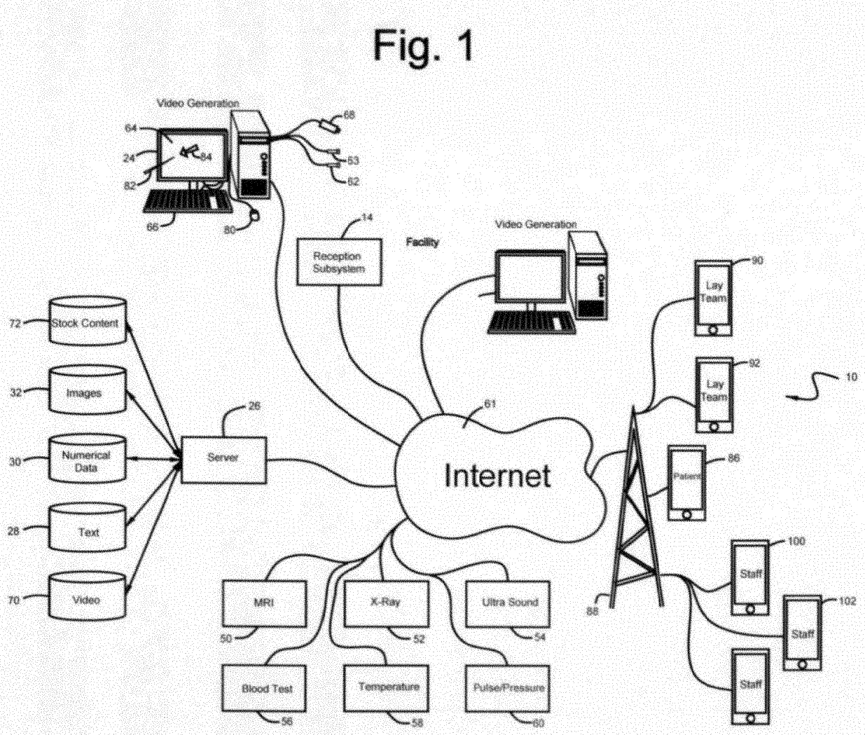

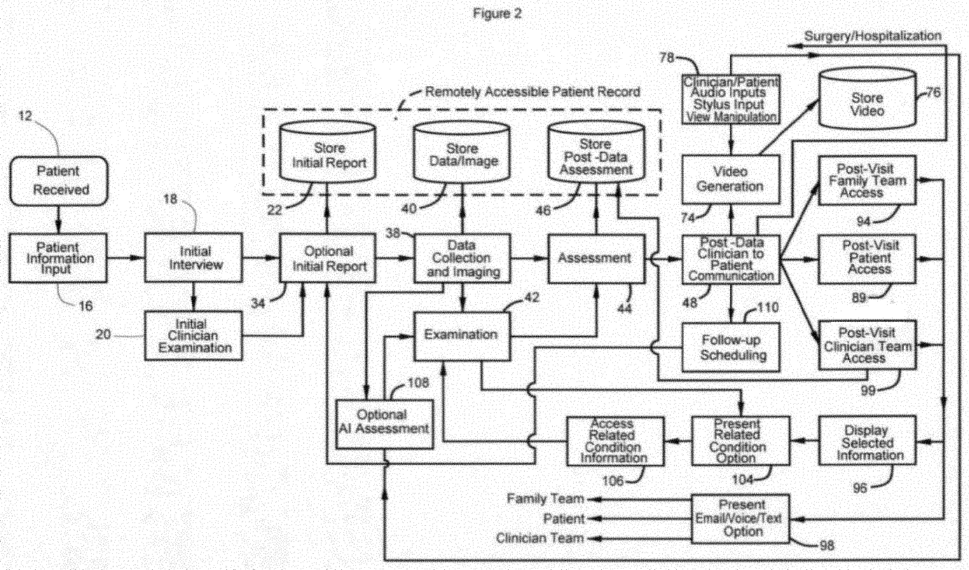

Referring to FIGS. 1 and 2, a hardware system 10 constructed in accordance with the present invention and suitable for practicing the method of the present invention is illustrated. Generally, in accordance with the invention, the inventive method may be initiated at step 12 (FIG. 2) by patients being received at the health facility, such as a hospital, where a reception subsystem 14 (FIG. 1) receives patient information at step 16 (FIG. 2). This collection of information is of the type normally collected by a healthcare provider.

For example, if the patient is new to the practice or the facility, detailed information may be collected including such things as allergies, existing conditions, symptoms, medications, and so forth. On the other hand, if the patient is known to the practice, less information may be collected as determined by the facility and/or practitioner.

After the information has been collected, the patient is initially seen by a clinician, such as a doctor, nurse, or other professional at step 18. At the initial interview, the clinician discusses the reasons for the visit with the patient in a manner determined by the clinician and consistent with current best practices in the healthcare sector. Such information discussed includes the reasons for the patient coming to visit the facility. In addition, the clinician asks the patient questions to gather information respecting the medical issue to be addressed.

At the same time, the clinician, whether a doctor or a nurse or other professional may conduct an initial physical examination of the patient at step 20. Initial information, both that collected orally and during the physical examination of step 20, may be stored at step 22 for the purpose of being assembled into an initial report by being input into a computer, such as a personal computer 24 (FIG. 1), which is in communication with a central server 26. Server 26, in turn, receives information for the initial report and saves it in an appropriate database, for example text database 28, numerical database 30 or image database 32.

In accordance with the present invention, it is contemplated that information from an initial interview with, for example, a nurse may be aggregated with the other information generated during the initial interaction between the patient and the clinician or clinicians, including information gathered orally, images, readings from instrumentation and other numerical data. The aggregated data can be then used at step 34 to augment the optional initial report which may be stored at step 22, and which may be provided to a doctor who might optionally direct further data collection and imaging at step 38. Such data and images, including those initially collected and those further generated as a result of a doctor's direction are then stored at step 40.

After the gathering of images and data at step 38, the information may be reviewed by the doctor who may elect either to do a supplemental interview and examination of the patient at step 42, following which the doctor may assess the situation at step 44 and store an updated assessment of the situation at step 46.

Alternatively, the doctor may elect not to further examine the patient and proceed directly to assessment step 44. After assessment has been completed at step 44, the doctor proceeds to meet with the patient at step 48 to discuss with the patient the various data collected, as detailed above, as well as other data as may be specified by the doctor or other clinician. Such data may include data collected at step 38, for example using an MRI device 50, an x-ray imaging device 52, an ultrasound imaging device 54, conventional blood testing equipment 56, a body temperature measuring device 58, or devices 60 for measuring blood pressure related parameters including systolic and diastolic blood pressures and pulse rate. These devices may provide output displays giving the test results, such as a touchscreen. Alternatively, or in addition, these devices may be wirelessly (for example by Bluetooth™ technology) connected to a computing device, such as a smartphone or PC, which relays the information over the Internet to server 26.

In accordance with the invention, it is contemplated that all information will be accessible through server 26. This is achieved by coupling all parts of the system to server 26 through cyberspace 61. This includes inputs from the various personnel, audio inputs, video inputs, stock, template or form inputs, pen inputs, and so forth.

During the meeting with the patient, the doctor's assessment of the situation is described to the patient. In addition, the doctor, in describing the situation to the patient uses video images and data generated earlier in the process, as described above. The contents of the interview may include a description of the condition, directions for treatment, drugs to be taken, instructions for taking the drugs, conditions, symptoms or other indications for the patient and for those persons associated with the patient on their layperson team, such as their family, to be on the lookout for (such as pain, visible changes, etc.), diet, limits on physical activity, recommended physical activities, and so forth as may be determined by the doctor or other clinician, such as a physical therapist, trainer, radiology treatment clinician, and so forth.

In accordance with the invention, the voice of the doctor and, optionally, the voice of the patient is recorded in a video which memorializes and makes a record of the same available to numerous individuals involved in the treatment of the patient.

In accordance with the invention it is contemplated that a video will be generated during the interview of step 48, and that the same video will include images related to the condition of the patient, prescriptions, instructions, and the like which go along with a running description given by the doctor as he goes through the various images, prescriptions, instructions, and the like detailing what is to be done, and giving the patient other useful information. This video becomes part of the permanent record which is accessible to the patient and their family after the interview, together with additional information associated with the patient, as more fully appears below.

More particularly, during the discussion held at step 48, the doctor selects, for example, from a menu of visual images, three-dimensional images such as patient x-rays, test results in text form, stock instructions, and so forth and explains to the patient the relationship of the same to the treatment plan for the patient. In addition, voice-recognition circuitry may be used to generate text specific to the patient's needs. Voice-recognition is responsive to microphone 62 (for example mounted on the lapel of the doctor's uniform) and may optionally generate text on the touchscreen 64 of computer 24. This text may be edited either using keyboard 66 or voice commands spoken by the doctor into microphone 62. However, software may be provided to display the finished text material from the beginning of the dictation to the end thus presenting it on the screen for an extended period of time and allowing the patient to study the same, for example after remotely accessing the same in accordance with in the invention.

In accordance with the invention it is also contemplated that the voice of the patient may, optionally, also be recorded. The same may be provided by a freestanding microphone, or by a microphone mounted on the collar of the patient's clothing. In this way the patient can ask their questions and hear the answers, and have access to the questions and answers after leaving the doctor's office, as appears more fully below. It is expected that this will increase the effectiveness of communication because patients often don't hear or fully understand what is being said to them during the interview and are reticent to take the time of the doctor by asking him to repeat what he said. Moreover, because the interview is available as a video, in accordance with the invention, after the interview is concluded, if the patient, upon hearing the question and listening to the answer again still does not understand the situation, he can initiate communication with the physician, using microphone 63, as more fully appears below. In addition, the communication is specific to a particular part of the video of the interview, and the availability of the same to the doctor, perhaps days later, allows precise information to be given to the patient by, for example, email.

More particularly, in accordance with the invention, it is contemplated that different patients may have different communications needs. For example, if the patient is not a native English language speaker, the patient may require instructions in another language, such as Spanish. In accordance with the invention, it is contemplated that the system will store such information as language preference, level of education, patient profession, specialized education of the patient, or other factors, or combinations of the same in order to develop a communications protocol, optionally utilizing artificial intelligence, which takes full advantage of patient capabilities and communicates in an effective matter regardless of the level of patient knowledge and communications ability.

In accordance with the invention, it is also contemplated that the doctor may wish to use a camera 68 in connection with explaining the condition to the patient and explaining what steps the patient should perform. Also, the camera may be used to show the patient what signs of danger or progress to look for. More particularly, in accordance with the invention, it is contemplated that camera 68 may be aimed at an area being treated, and the image from video camera 68 may then be processed by computer 24 and ultimately stored by server 26. The camera 68 may also be used to capture an image of any item of interest, such as a paper prescription. Camera 68 may also be used to capture a video of a desired procedure, such as a procedure to be carried out by the patient for equalizing pressure in the ear, as might be carried out without equipment or with a device such as the pressure equalizer sold under the trademark EarPopper. More particularly, it is contemplated that the entire interview will result in the production of a video which will be stored by server 26 on video database 70 for access by the patient and other users of the inventive apparatus. It is contemplated in accordance with the present invention that the doctor will use language appropriate to the health literacy of the patient to explain the medical issues and instruct the patient. This will also serve the interest of clear communication with lay persons on the patient's team, such as family, translator, health care advocate, etc. It is further contemplated that the doctor will also address the issues and give directions for future action such as drug administration and exercise in a way that will also give clear direction to persons on the professional clinician team, such as nurses, physical therapists, physician specialists, and so forth.

In creating the video at the interview, the doctor may also rely on stock content stored in memory 72, incorporating such stock content into the video being generated at step 74, simultaneously with the conducting of the interview. When the interview is complete, the video is stored at step 76. As noted above, the outputs of microphones 62 and 63 and camera 68 are sent at step 78 to computer 24 for video generation at step 74. Further in accordance with the invention, much of the video may be pre-prepared in the form of the template, for example by a nurse, and the doctor may supplement, select from options, and otherwise use the pre-prepared template as a vehicle to build time efficiency into the interview process.

In accordance with the invention, the inventive apparatus may accommodate a library of selectable content generated by the healthcare provider or the healthcare system to which the healthcare provider belongs. The same may optionally be accessed through artificial intelligence or, alternatively, it may be manually accessed. In addition, in accordance with the invention, it is contemplated that the system will generate a diagnosis/treatment protocol based on input information as a mechanism of profiling patient information needs, optionally for presentation to the healthcare professional. Optionally or alternatively, this protocol may be used as an input to a search algorithm which locates existing informational resources on the Web for convenient and efficient presentation to the patient, allowing the patient to better inform herself or himself respecting the condition, with the objective of accommodating the need of patients for information in order to make them comfortable with their treatment from a psychological standpoint, and also to educate patients and build their ability to communicate information to their treatment team. In one embodiment, the information being made available to the patient may be limited to information in a library maintained by the user of the system. In addition, while the library may be accessed using an artificial intelligence algorithm, the invention also contemplates the presentation of resources to a treatment team member for dragging and dropping into a mailbox accessible to the patient. Moreover, the invention further contemplates an embodiment where initial searching for information is performed by a search engine, and the initially presented innovation is then selected by a human operator, for example the physician's assistant or a surgeon.

In accordance with the invention it is also contemplated that the doctor may, optionally, prepare a base video prior to seeing the patient, for example during the assessment at step 44. That base video is then played back during the patient interview of step 48. During the patient interview of step 48, the base video may be played back and modified by the addition of material and/or by the removal of material. Optionally, the base video may be prepared by a support staff member (such as a nurse, physician's assistant or technician, or a combination of such persons) for modification and finalization by the doctor or other principal clinician (such as a dentist, physical therapist, psychologist or other health professional).

When the doctor meets the patient at the interview of step 48, the doctor or other principal professional can go to the base video in sequence (or out of sequence) at a rate which is fast or slow, as required, and add as much explanation and take as many client questions from the patient as the principal clinician deems appropriate. Likewise, the order of the elements in a prepared video may be varied before, during or after the interview of step 48.

However, once indicated as finalized by the doctor, the video becomes immutable and, because it records the actions taken by the clinician, can serve as an excellent tool to protect the institution, doctor, and others against potential legal liabilities due to malpractice, alleged misunderstandings, and so forth. The created video may be made immutable by locking the video file against editing and placing it in an access restricted folder. The accuracy of the video as an unchanged record may further be verified by looking at the properties of the video file as well as any other metadata which may be added to the file for security purposes. Likewise, the videos created in accordance with the invention are also useful as a means of monitoring the quality of service provided by individual clinicians.

In accordance with the invention it is also contemplated that the doctor may elect not to include portions of the patient interview of step 48 in the video, such as re-explanations and conversation meant to verify patient understanding. Likewise, a confusing segment of conversation may be edited out of the video during the interview of step 48, or this may be done after the interview of step 48.

When it is desired to use the inventive system 10, for example, a patient may be given an initial interview at step 18 resulting in the storage of collected information by server 26 in the appropriate database. This information is then available later on in the process. After the initial interview, data may be collected by equipment such as an MRI machine 50, x-ray machine 52, ultrasound imaging devices 54, conventional blood test equipment 56, a thermometer 58 for measuring body temperature, and blood pressure parameter equipment 60. This information will be transmitted over cyberspace 61 to server 26 and stored in the appropriate databases, such as text database 28 or image database 32.

Such stored information is then available for use by a doctor in making an assessment at step 44. This assessment may be followed by or done simultaneously with the creation of the video at step 74. In the creation of the video, the physician may, for example, access a three-dimensional image of an affected area of the body of the patient, such as the lung in the case of a pneumonia patient. Using a mouse 80, the physician may manipulate, for example, an MRI image in three dimensions to obtain a desired view. As the image is being manipulated, the image is being recorded by the system in real time, allowing the patient to see, for example, in the beginning, the entire area imaged and then allowing the doctor to zoom in on a particular area for examination and explanation to the patient. As the image is being zoomed in on and being manipulated, it is recorded, and thus incorporated into the video to be made accessible, after the interview, over the inventive system to the patient and to others on their care team, including their doctors, outside providers and family. Once the doctor visualizes on the screen an image area with respect to which he would like to discuss the condition with the patient, the doctor may take a stylus 82 and use it on touchscreen 64. Using stylus 82, he may, for example, point out in the area on the lung where infection is visible (optionally drawing a ring around it) and explain the condition to the patient. This explanation is recorded by microphone 62 and included in the video, as is the ring drawn by the doctor. In addition, if the patient asks a question, the question is recorded and included in the video through the use of microphone 63. Likewise, the position of the stylus on the touchscreen may be indicated on the touchscreen by an appropriate visual device, such as an arrow 84.

In accordance with the invention, it is contemplated that everything displayed on touchscreen 64 is recorded as a video for later reference by the patient, family members, personal friends and perhaps others involved in the treatment, and the doctor and persons working with the doctor in a clinician care team. In the case of some conditions the clinician care team may include a number of specialists, such as a cardiologist and a neurologist. Also, on the clinician care team are nurses, technicians and other specialists working in, for example, a hospital or an out of hospital treatment facility, such as a dialysis facility.

In addition, as alluded to above, camera 68 may be used to visually display on touchscreen 64 an image of a part of the patient's body. For example, if the patient is being treated for eczema, camera 68 may be aimed at the affected area and the doctor may explain the situation while using stylus 82 to create the display of an arrow to show principal features of the area of the skin affected with eczema. In addition, the stylus may be used to encircle an area, as if a pen were being used on a paper image, to describe its size or extent. The doctor may also explain whether certain areas of the affected body part might develop other appearances and that the patient should be on the lookout for improvement or, possibly visual symptoms of complications, as well as sensory indications of complications, such as pain. This information can be explained by the doctor using microphone 62 to include such information on the video. If desired, the software may also provide means for a note to be tacked onto the screen and for the doctor to type it an alphanumeric instruction or list or other type of communication into the note using keyboard 66. Optionally, a template for the video may be used and the template may present an area for the inclusion of such alphanumeric information, for example as a blank slide, such as a PowerPoint™ slide, which may take up all or part of the screen.

As alluded to above, the record being created by the video is available to the patient and persons on their personal care team (such as family members) and their clinician team members, as appears more fully below.

In particular, the patient may use their smartphone 86, connected by their ISP provider's system 88 to cyberspace 61, to access, at step 89 the video created during the communication conducted at step 48 and turned into a video at step 74 for storage at step 76. The screen of the smartphone of the patient may present an icon for the retrieval and display of the video, as well as icons for retrieving prescriptions, instructions, and related condition information. Such access is done by a dedicated application which has been downloaded onto smartphone 86. Likewise, the lay persons on the personal care team of the patient may use their smartphones 90 and 92 to access the video at step 94.

In accordance with the invention, the healthcare record may be segregated into multiple sectors. For example, one sector can be devoted to information which can be made accessible to the patient, or perhaps the patient but not members of their personal care team. Other sectors may be limited to other healthcare providers. In this case, the language used and the descriptions given in the “healthcare provider sector” would be tailored to professionals, efficient communication and other needs of clinician to clinician communication. All types of media may be used. Pictures, audio, images video etc. Such information may also take the form of an audio file.

In accordance with the invention, the provided app can also display selected information, such as prescriptions, drugs, instructions and the like and upon the institution of a request for such information by the application, the same is displayed on the smartphone of the person requesting information at step 96. Individuals using the application are also given the option of communicating with each other or clinician team members through email, voicemail, text or other communications options at step 98. Upon exercising the option to communicate the names of various team members both lay and professional would appear on the screen automatically for selection as recipients of the communication.

In accordance with the invention, a notifications panel is provided in the app downloaded on the smartphone of the patient, family or personal team lay member, or clinician team member. The notifications panel may be accessed, for example, by the healthcare provider and the patient. The patient and healthcare provider are directed and/or reminded to do this at an appropriate frequency. When the notifications panel is accessed, new information is highlighted and clicking on the appropriate icon will result in the smartphone navigating to the particular new element. It is also possible that there may be several new elements, perhaps from different people. In this case, the notifications would show a number of options corresponding to the same.

When the notification icon is touched, the patient or other user is given the information to read. When the review of the information is completed, the user then may touch a back icon and go back to the prior list or collection of icons. On a list the listing or icon of the original item is grayed out and the remaining items which have not yet been selected and reviewed are still bright, thus ensuring that the user has covered all notifications. If there is an emergency situation, notifications may optionally be supplemented with Robo calls, text messages, emails, or all of the same.

In connection with graying out notifications which have been accessed by patient, the clinician, etc., it is noted that those notifications will be made available to other members of the team who can see, judging by which items have been greyed out in a different color, which items have been reviewed by the patient or other team members. For example, a yellow color may indicate a patient review, and a gray color may indicate review by the patient and a professional team member.

Another feature of the app used by the patient is the ability for the patient to add an individual and/or designated healthcare proxy, to their, for example, home/personal care team. Any appropriate verification procedure may be employed to be sure that this is a proper addition.

When a patient on boards a medical professional, the individual needs to be authenticated as a medical professional. In accordance with the invention, this may be done by checking against a database, or by a human using manual techniques.

In the event that an individual from the clinician team is sent a communication, that individual may access the communication on his or their smartphone, for example smartphones 100 and 102. Clinician team member smartphones are provided with their own app for accessing the video, and other information stored by server 26. Clinician team members are also provided with a communications option at step 98. In connection with such communications, clinician team members may choose to access data stored by server 26 at step 99, resulting in display at step 96. In accordance with the invention it is contemplated that clinician team members and the patient will have access to parts of the video or other documentation, images and the like and that the same may be referenced by doctors, a layperson team members and/or the patient in making a communication, for example a communication seeking to instruct the clinician team member or ask a question of the clinician team member. Accordingly, the quality of communication is may be enhanced by the present invention.

In accordance with the invention it is further contemplated that images (or other information) being viewed by the patient, clinician team member, personal care team member or other persons accessing the system may be marked for inclusion in the next email to be sent by the system. This marking would be transmitted to the central server and would enable the central server to send these marked up images along with the alphanumeric, voice or other communication to the clinician team member, for example where the person initiating the communication is a layperson, to provide easy access to the clinician team member receiving the question or information communication from the lay team member. Likewise, it is contemplated that the same mechanism may be used in the case of communications being initiated by the clinician team member and being communicated to other clinician team members or the patient or patient personal team members. Likewise, such mechanism may be used in the case of communications between lay personal team members.

In accordance with the invention, it is further contemplated that, optionally, a display of selected information at step 96 may also result in the display of icons at step 104 providing the option of access to information related to related conditions. If the icon is clicked by the individual requesting such related information, the information is provided at step 106. This information may also be provided to the doctor at step 42 during the doctor's examination.

In accordance with the invention, it is yet further contemplated that, optionally, artificial intelligence responsive, for example, to the position of the stylus on the screen as it is being manipulated by the doctor may be employed. More particularly, because the doctor is putting the stylus on the screen in a particular area of an image, the system may use an artificial intelligence algorithm to evaluate the image compared to typical images and/or images associated with a large number of conditions, in order to assist the doctor in a diagnosis at an optional artificial intelligence assessment step 108. The AI system could, after the doctor does his or her analysis work, present a series of images to the doctor for study along with text indicating the reason for the flagging of the images and other information determined by the AI system to be of interest to the doctor.

The system AI algorithm may be made sensitive to symptoms noted by the doctor and input into the system from a list of symptoms. These symptoms may be input into the system by presenting a diagram in the form of a tree for navigation and selection by the doctor. Such a tree at an initial level may present head, torso, left and right arm and left and right leg options, with each of these including suboptions such as thigh, knee, calf and other options in the case of a leg selection by the doctor, and further increasingly specific options, such as a) pain, bruising and cuts, b) severity of pain, and so forth, and the like.

The AI algorithm may be made to monitor the amount of time that the doctor spends looking at a particular area on an image during an analysis conducted by the doctor prior to the patient interview, and on the basis of the time spent in active observation of an area do extensive image and symptom checks on that area or on the symptoms noted by the doctor. This information can be provided to the doctor at, for example, a subsequent examination or provided in real time to the doctor while he is generating the video, for example in an inset screen on touchscreen 24. In this case, the inset screen would not be part of the video. It is expected that such artificial intelligence input may be of particular value if it is also made responsive to subsequent team communications and provided at a subsequent visit of the patient to the facility as scheduled at step 110.

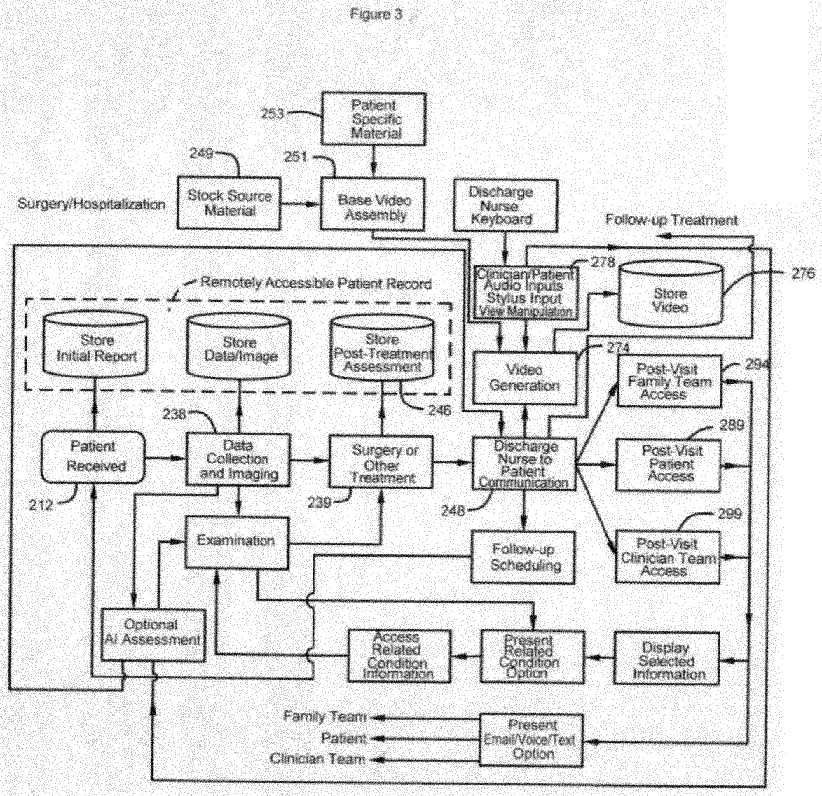

In accordance with the present invention, it is contemplated that similar methodology will be employed in the case of the discharge of a patient after surgery. The steps of this aspect of the inventive method are illustrated in FIG. 3 and deal with the method for the execution of surgery on a patient who has returned to a hospital, for example, for surgery. The method illustrated in FIG. 3 is in many respects similar to the method outlined in FIG. 2, as can be seen by a comparison of the illustrated method steps. To the extent that there are significant variations, the same are described in detail below.

More particularly, in accordance with the invention, the patient is received at step 212 for purposes of surgery. At step 238 data collection and imaging may, optionally, be conducted. Surgery is then performed at step 239 after which there is a posttreatment assessment and storage of that information at step 246.

After the surgery, the patient is sent to discharge where the patient is seen by the discharge nurse at step 248. In accordance with the present invention, the discharge nurse gathers stock material at step 249 and begins to put together a video at step 251 using computer 255 (FIG. 1). At step 253 that video includes patient specific material and contributes to the generation of a video at step 274. That video is then used as a base during the discharge at step 248. However, in accordance with step 248 the video used is the pre-prepared video created by the nurse at step 251. However, it is augmented by stylus, audio, keyboard and other inputs at step 278. The completed video is sent by cyberspace to server 26 which stores the same in video database 70 for later access by the patient and team members.

Once created, the video (including the base video generated at step 251 as modified at step 278 and stored at step 276) is available to various team members at steps 294, 289 and 299, for use as described above in connection with FIG. 2.

In accordance with the invention, the primary use of the video, is to make the same available to the patient. Often patients have trouble remembering what was said, or understanding everything which was said. Indeed, a patient may leave a post-operative or post office visit interview/discussion thinking he or she heard and understood everything, and that just may not be the case. The possibility here is that the patient will neglect to do something which should be done or that the information might be miscommunicated to those around the patient, such as family members, and cause problems or other complications.

In accordance with the invention, the video with the healthcare professional is posted on the web and, once posted, is accessible using a PC, laptop, tablet and/or a smartphone. This enables communication of the circumstances surrounding the health problem to family members, employers, partners, and so forth to the extent that the patient wishes to share the same information. At the very least it is a detailed memorandum to the patient which can be reviewed and reviewed again until the patient is satisfied that she or he knows what has to be done or, alternatively needs to ask certain questions in order to meet the responsibility of taking care of herself or himself.

In accordance with the invention, it is further contemplated that the creation and supplementation and later augmentation of the inventive video as described herein may be done, for example, in consultations with family, where discussions with the family would be added to an existing video or used to create an additional law video. For example, caregivers in the home should be aware of what is happening and what they should be doing to help. The doctor may call the family and, even when the patient is not present, and explain potential issues and what has to be done. The family, at the same session can ask questions and answers. At the same time, the doctor is essentially creating a video including all materials which he gathered for the discussion with the family and all of the dialogue between the clinician and the family. This would then be joined to the existing record available on the website. In accordance with the invention, it is contemplated that the individual parts of the record may be shown, for example, as a menu of images on a smartphone. The patient, family, etc. can then use their smartphone, look at the menu of images, click on the one covering the subject that they want to learn about, listen to it and then move on to another one if they so choose.

At this point, it is contemplated that the proper approach would be for the doctor or other clinician to act as a gatekeeper with respect to new content. However, it is expected that patients will be encouraged to communicate with the clinician, using text and perhaps even sending a photograph of, for example, a wound in the process of healing in order to get further guidance with respect to future treatment of the condition. Once this information is sent to the clinician, the clinician may elect to add it to the patient record. All of this information, once it is loaded onto the system will be available to the patient. Thus, it is contemplated that the application will have icons on the smartphone screen which will create a picture and send it automatically to the doctor, create a window for the entry of text to be sent to the doctor, initiate an email and the like

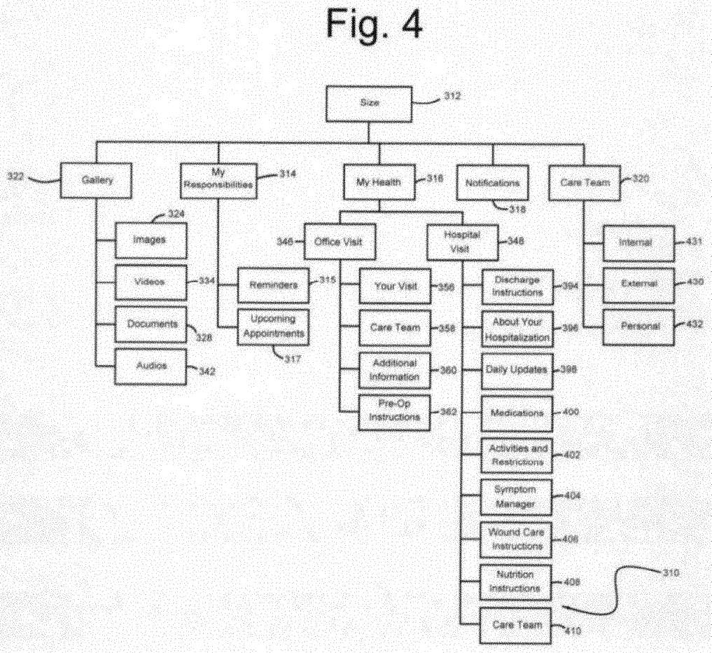

Referring to FIG. 4, a method 100 carried out in accordance with the present invention is illustrated. More particularly, FIG. 4 represents an application of the inventive method to a mobile device, such as a device operating on the latest iOS operating system, and constituting an exemplary embodiment of the method of the present invention, which may be implemented on the IT infrastructure illustrated in FIG. 1.

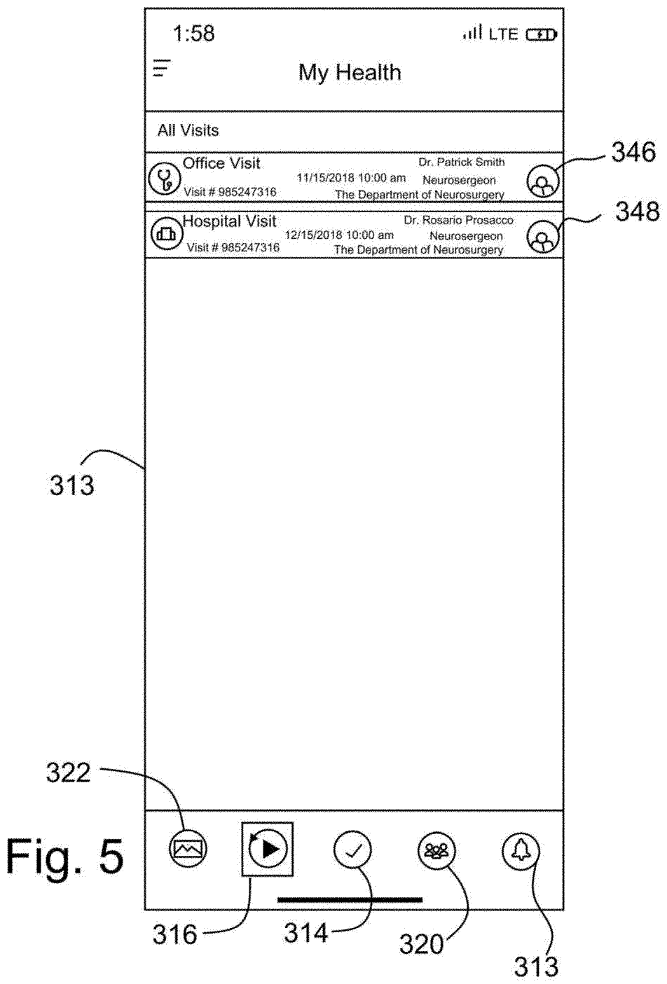

Referring to FIG. 4, in accordance with the present invention, the inventive method 310 may be initiated at step 312 by patients who have installed a mobile app on their smartphone or other mobile device. The mobile app is structured to implement, on the mobile device, the method illustrated in FIG. 4, as is more fully explained below. More particularly, in FIG. 4, method steps are given descriptive designations meant to provide a general overview of their functionality. In addition, where practical, numerical descriptors corresponding to icons in the graphical user interfaces (which are illustrated in FIGS. 5-30 and described below) which enable use of the patient app by the patient to present and transmit information.

More particularly, the patient may use his or her smartphone 313, which has had the app downloaded onto the smartphone, to access all information generated for, collected from and otherwise associated with the patient in accordance with the general methodology disclosed in connection with the description of FIGS. 1-3. Such information is generally included in the patient's record of a particular patient-user of the inventive app. The inventive system may also have collections of facility specific information meant for use by multiple patient users. As discussed above, this may include video and audio records of such things as patient and doctor interactions, physical examination, doctor notes, and so forth.

All the information collected relating to the patient is stored in databases 28, 30, 32, 70 and 72 via Internet 61 and server 26. These databases may be accessed by the user using their smartphone 313 by using the inventive mobile app which was downloaded to the smartphone of the user and which implements the inventive method 310.

In particular, when the user wishes to access information, the user boots up the inventive app at step 312 and is presented with the touchscreen display of FIG. 5, offering options for accessing information in, for example, five different categories. These categories may include, for example, 1) information relating to patient responsibilities accessible at virtual touchscreen button 314, 2) the status of patient health and treatment (including upcoming appointments) accessible at virtual touchscreen button 316, 3) notifications to the patient accessible at virtual touchscreen button 318. Such notifications might typically require patient attention or keep the patient informed. Likewise, buttons may optionally include 4) access to and information respecting the patient's care team accessible at virtual touchscreen button 320. Likewise, 5) a “gallery” of images, videos, documents and audio recordings may be accessed through another category option accessible at virtual touchscreen button 322.

More particularly, after booting up the application at step 312, the user is presented with a screen, such as that illustrated in FIG. 5, which presents options corresponding to the above five categories but defaults to one of the categories, in this example, treatment associated with two treating physicians, which grants access to associated information as detailed below.

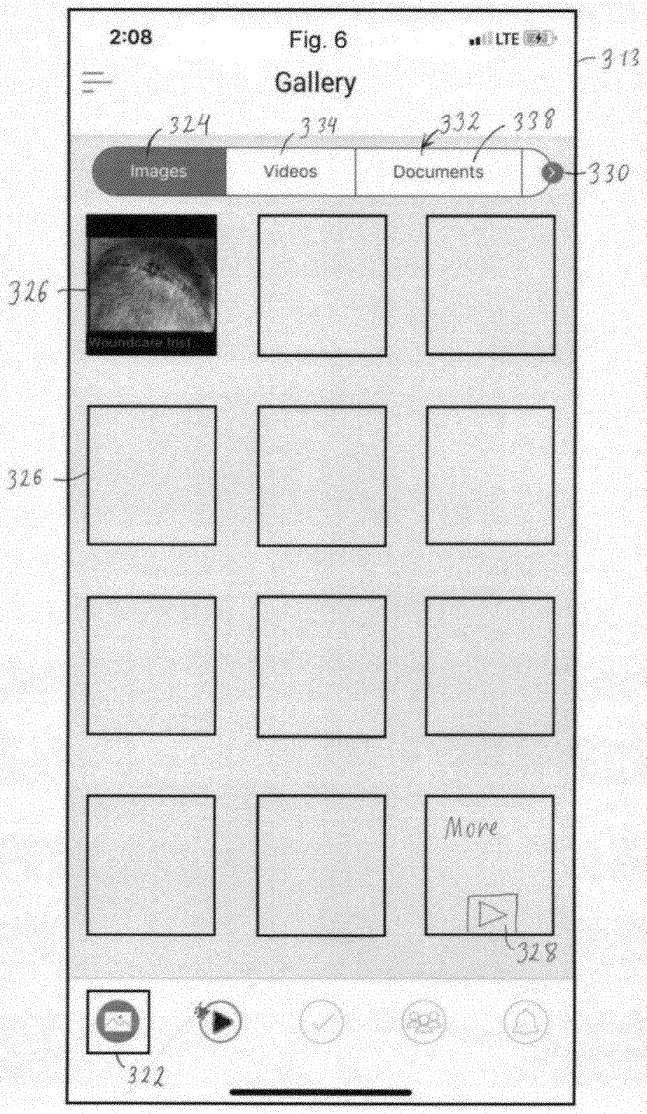

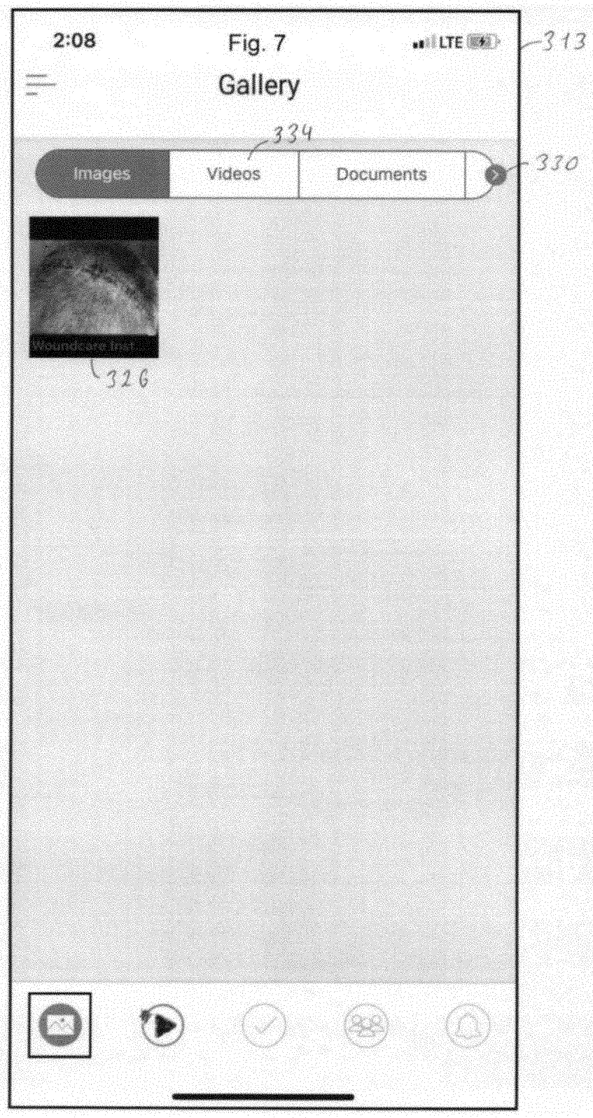

Tapping gallery button 322 presents access to information in forms such as pictures, videos, documents and audio recordings, for example in a graphic user interface such as that illustrated in FIG. 6. More particularly, pushing button 324 presents icons 326 representing images. Tapping on icon 328 brings the user to additional materials, as illustrated in FIG. 7. Tapping on one of the icons 326 brings up the image resource associated with the particular icon.

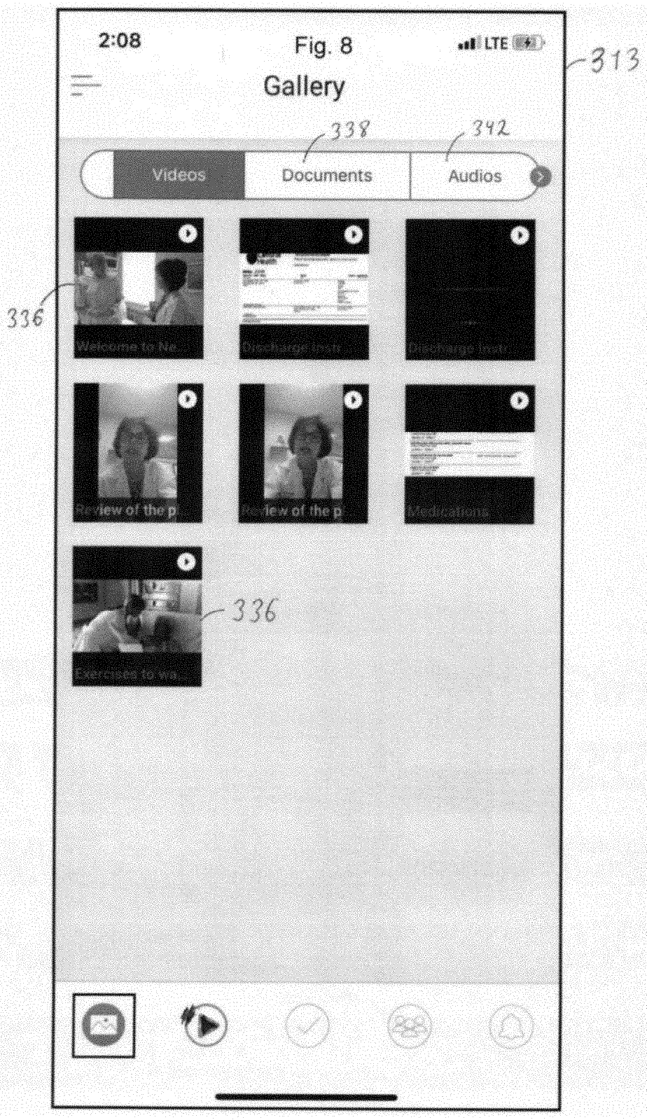

Tapping on icon 330 in the screen of FIG. 6 shifts the contents of menu 332 to that illustrated in FIG. 7. Conversely, pushing button 334 in the screens illustrated FIG. 6 or FIG. 7 brings the user to the graphic user interface illustrated in FIG. 8, where videos accessible to the patient are indicated at icons 336. Tapping on icon 336 brings up onto the screen of the smartphone the display of the video associated with the icon. The patient may watch this video in a conventional screen including such features as play, stop, high-speed scroll with preview, go back 15 seconds, go forward 15 seconds, and shift between full-screen and small screen displays. It is contemplated that the screen will adapt to maximize the size of the display and properly orient the image in response to vertical and horizontal orientations of the smartphone. In accordance with the invention, it is also contemplated that the icon 336 representing the video would comprise a representative frame from the video, optionally, was that by the doctor or other caregiver, on the basis of, for example, importance or other selection criteria, for example, a high likelihood of being forgotten.

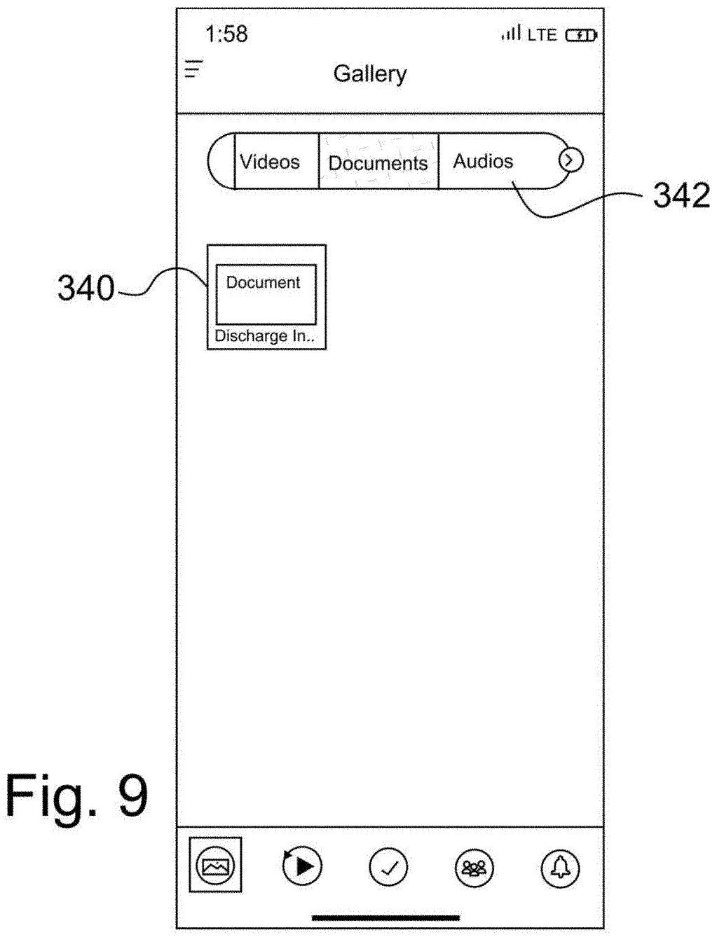

The patient may access documents by tapping on button 338 in any of the screens of FIGS. 6-10, for example. Tapping on button 338 brings up the screen of FIG. 9. One or more icons 340 are illustrated, each of the icons 340 representing a document related to the medical care being received by the patient. Tapping on icon 340 brings up a full-page display of the document (or a reader view) which may then be read by the patient.

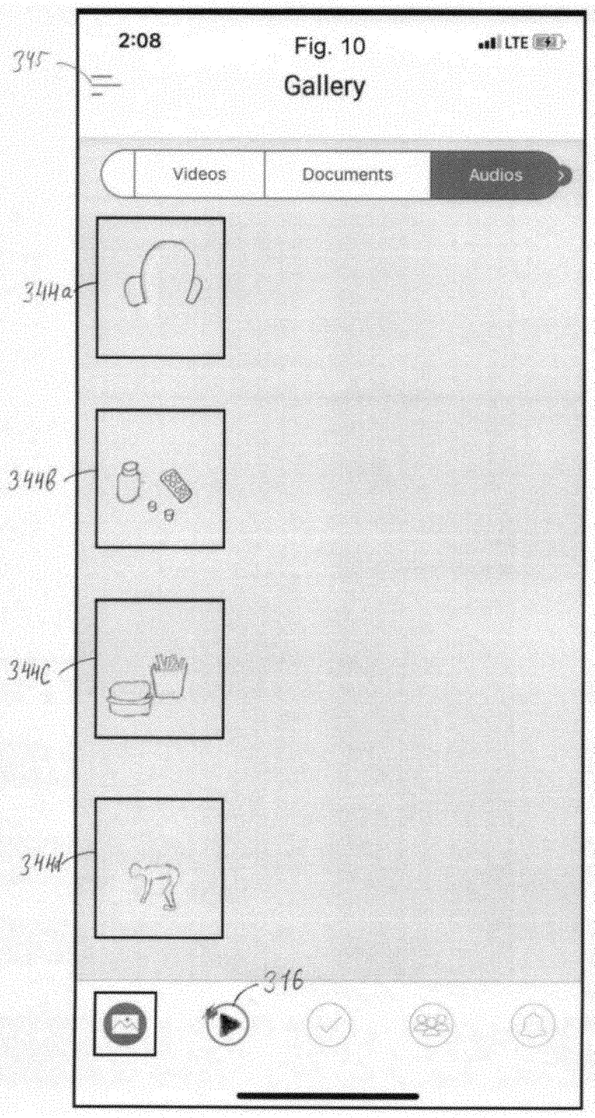

Tapping on audio icon 342, for example in the screen illustrated in FIG. 9 brings up the screen of FIG. 10. The graphical user interface illustrated in FIG. 10 presents the user with the option of playing various audio recordings by tapping on a respective icon 344 a-d. These icons, like other icons in the app may be of a general format, or may comprise an illustration which indicates their functionality. For example, icon 344a gives a general indication it is an audio by the illustration of a pair of headphones. On the other hand icons 344 b-d indicate their functionality with illustrations of a pill bottle and pills to indicate medications, an illustration of a burger and fries to indicate dietary guidelines for the patient, and an illustration of a person exercising to indicate physical activity recommendations, respectively.

At any point, icons 314-22 may be accessed by tapping. Upon the execution of the tap the user, the app returns the user to the selected category. More particularly when the app returns to the selected category, it will go back to the screen last viewed by the user in that category. Accordingly, for example, clicking on icon 316 in FIG. 10 will return the user-patient to the graphical user interface illustrated in FIG. 5. In the illustrated example, an office visit with Dr. Smith is indicated at location 346 on the screen, while a hospital visit with Dr. Prosacco is illustrated at location 348.

Similarly icon 345 illustrated in FIG. 10 can be accessed at any point. More particularly, by tapping the icon 345 user can view user profile that includes user name, picture, address, phone number, email and web site if applicable, and account information such as login and password. All the information related to user profile and account, can be edited by user.

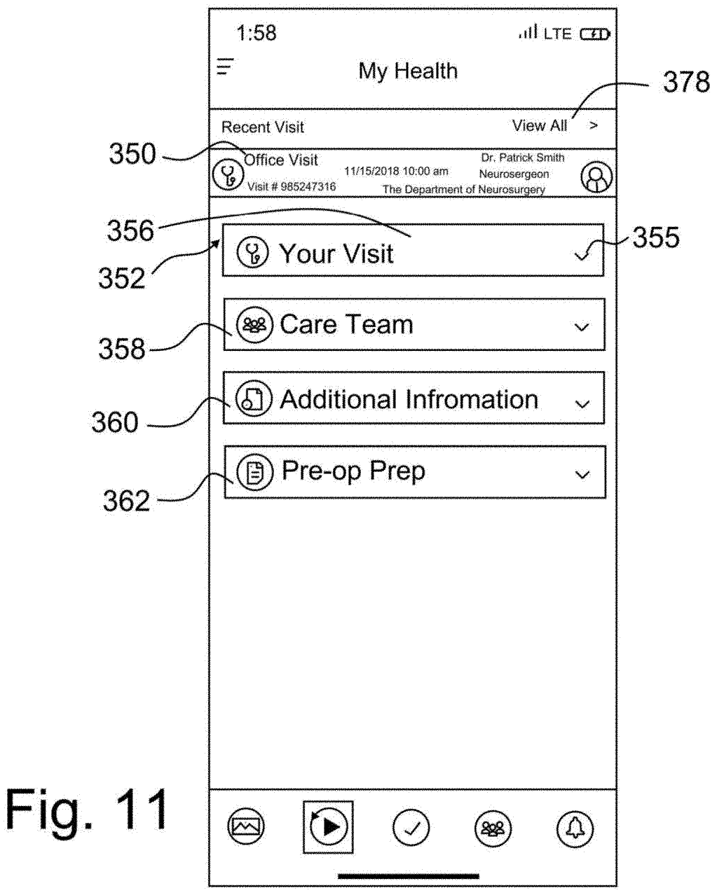

By clicking on the words “Office Visit” at location 350, icons 352 for accessing information relating to the office visit with Dr. Smith are presented to the patient, for example, in the form of the graphic user interface of FIG. 11. Such information may relate to a recent visit or an upcoming visit and the same may be indicated on the graphic user interface, for example at location 354.

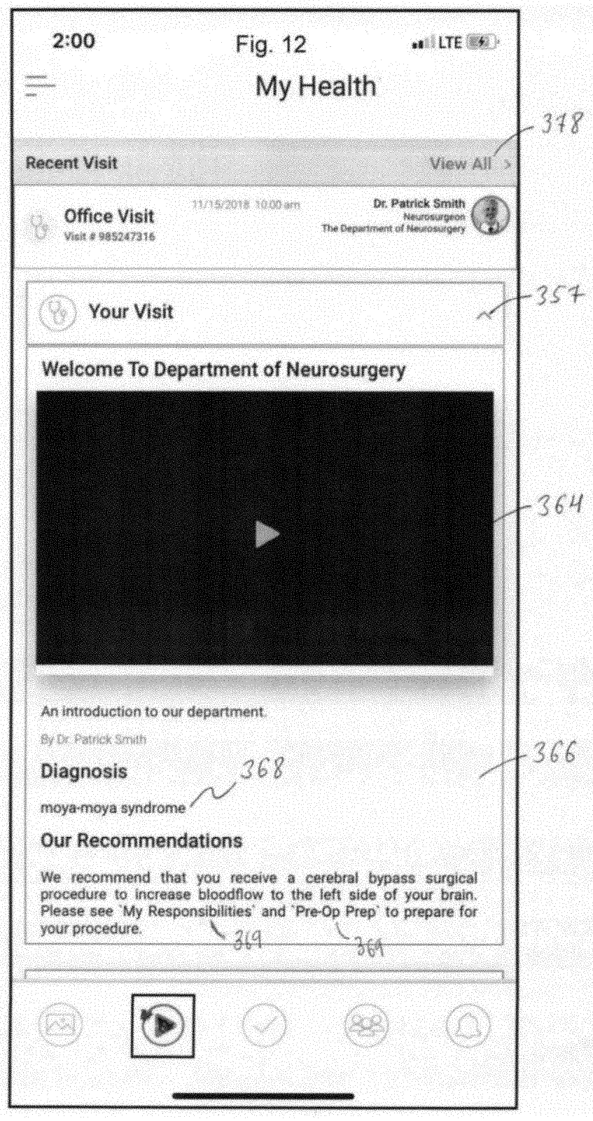

As can be seen in FIG. 11, multiple categories including office visit, your visit, care team, etc. are presented. By tapping on the appropriate icon, information respecting each of the same is presented. Alternatively, one may tap on the more information icon 355 associated with a particular category of information, which will cause the system to produce a display giving access to such more information. See for example FIG. 12 where icon 355 has been replaced by icon 357. If all the information cannot be displayed on the single screen, the part that is invisible can be viewed by scrolling up and down. All other icons can also be viewed by scrolling up and down while icon 356 is expanded.

The information access options for Office Visit corresponding to icons 352, as illustrated in FIG. 11 include information such as the dates of office visits, visit number, name of the doctor, department. Information is accessible through Your Visit icon 356, Care team icon 358, Additional information icon 360, and Pre-op Prep icon 362.

More particularly, tapping on Your Visit icon 356 brings up the information screen illustrated in FIG. 12. In the example, tapping icon 356 presents a screen with an icon 364 accessing a video presenting information respecting general information on the department of neurosurgery to the patient user. This introduction to the department video may give general information on the personal and facilities available, as well a specific information relating to the condition of the patient. Information specific to the patient may also be presented at location 366 and cover diagnosis, and recommendations such as necessary surgeries, procedures and other doctor visits.

Information indicator 368 may hyperlink to information on the Internet, such as web pages of WebMD™. Other information indicators 369 may link to information accessible through the inventive app. Such information may be information on the patient record, or more general information relating to the facility operating the app for the benefit of the patient user and/or meant for use by multiple patients.

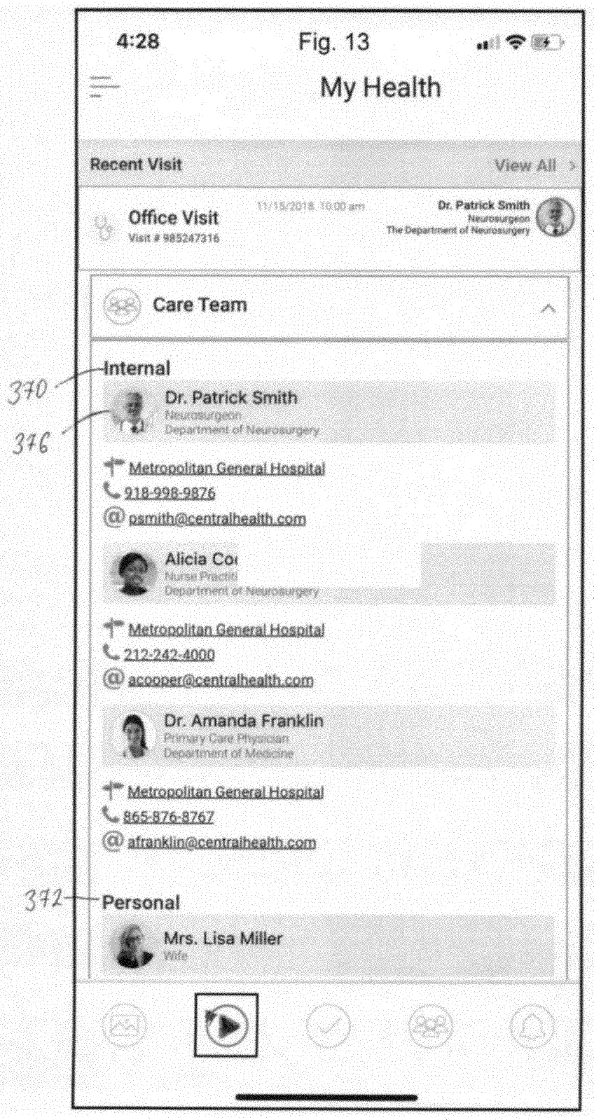

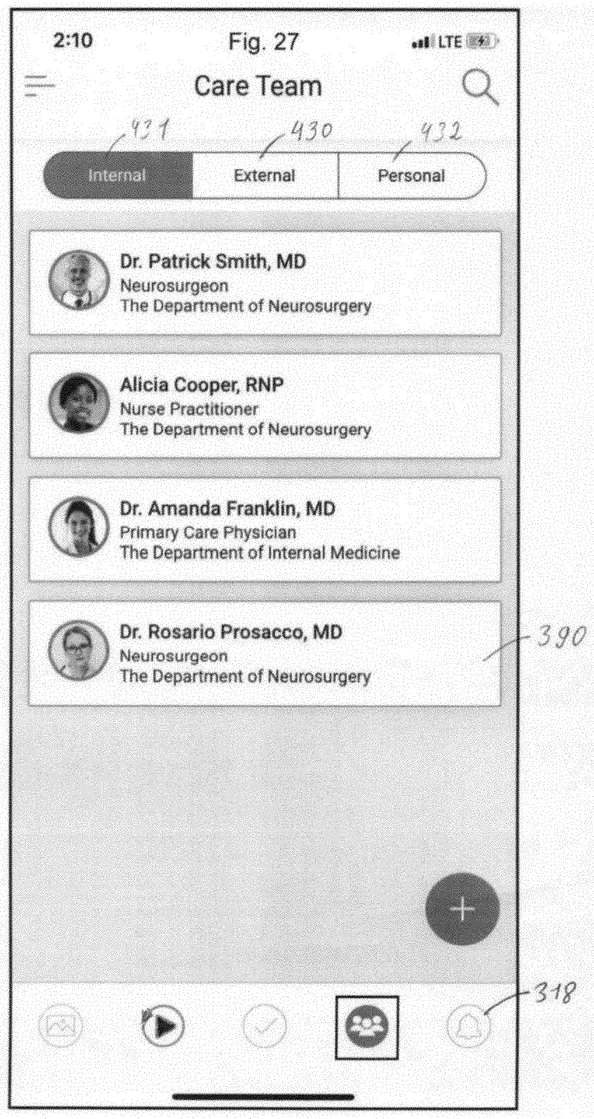

Tapping on care team icon 358 brings up FIG. 13. FIG. 13 presents a screen identifying members of the patient-user's office care team (in contrast with the screen of FIGS. 27, 29 and 30 which display complete lists of care team members by category), optionally, divided, for example, into three different categories: internal, external and personal/family, as illustrated in FIG. 13. Care team members are presented in three categories in the illustrated example. If all members cannot be seen on a single screen, an arrow icon may be used to scroll to additional care team members across, or up and down on, the display. Alternatively or additionally, the patient-user may be presented with a display of team members in a category by clicking on the category icon, such as internal team icon 370 or personal team icon 372. As illustrated in FIG. 13, the display presents, for each member of the care team, information such as name, office location, office phone number and email.

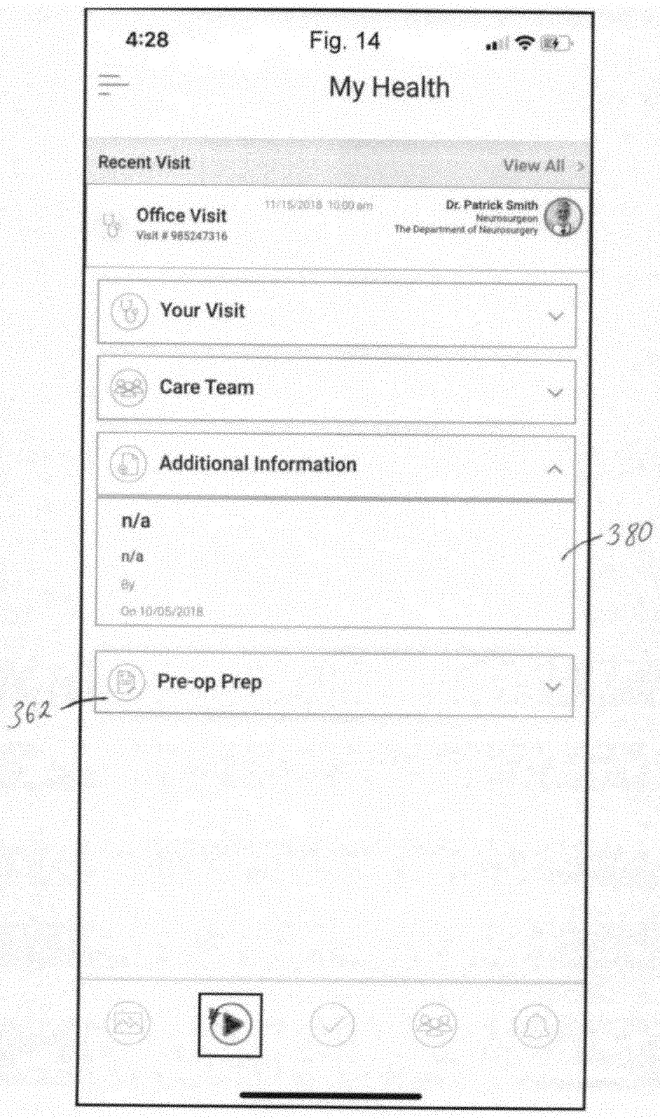

Optionally, by clicking on icon 376 associated with Dr. Smith, in FIG. 13, information (optionally viewed as of a more critical nature and thus included on numerous screens) respecting the care provided by Dr. Smith is provided, for example in the graphic user interface illustrated in FIG. 14. Alternatively, icon 376 may display more contact information of the doctor such as cell phone number, email address, location of work and so forth.

By tapping on the “View All” icon 378, for example in FIG. 12 or any of the other screens, one can return to a selected overview screen, for example the opening screen of the app, such as FIG. 5. More particularly, it is contemplated that the functionality of providing quick access to an overview from any screen would facilitate patient access in a single screen to all information by way of major information navigation icons 314-322 and critical information in particular, by listing the same on a single screen, and making that screen available from many if not all of the screens is a feature of the invention likely to have a beneficial impact on patient outcomes. Thus, the likelihood of a patient missing critical information can be drastically reduced pursuant to the invention. In this fashion, adverse effects on health of the patient can be reduced and patient outcomes improved. Such critical information may be selected by one or more members of the professional care team and/or the patient, and may include upcoming appointments, treatments, or other aspects of treatment deemed more important.

Tapping on additional information icon 360, for example in FIG. 11, brings up in an information field 380 additional, optionally less important, information put up by the professional staff, such as symptoms to watch out for, overall information optionally linked to information on the web, a change of member in care team, a doctor being unavailable and so forth, in a screen such as illustrated in FIG. 14. In the event that no such information has been input by the staff, an “n/a” or not applicable indicator would be presented, as illustrated.

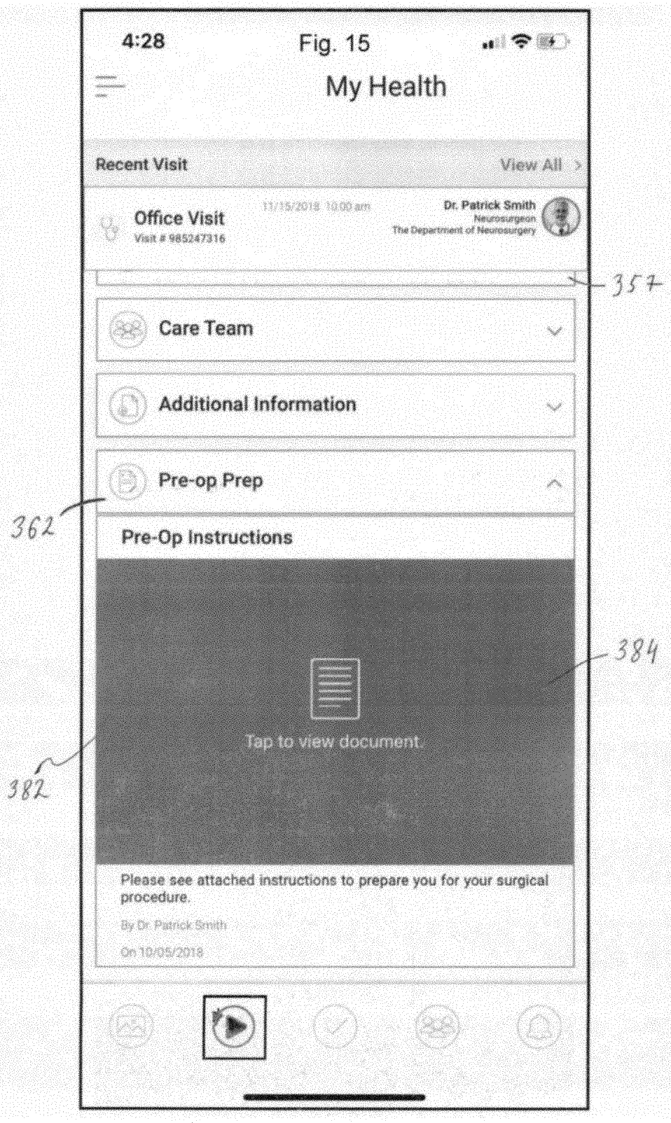

In accordance with the invention, patients about toward the core procedure would have information entered into the system. This specialized information relating to the procedure and preoperative preparation and procedures may be accessed by clicking on icon 362 which appears, for example, in the screen illustrated in FIG. 11. The result is the presentation of the screen illustrated in FIG. 15. It is noted that the screen of FIG. 15 may also be accessed by clicking on icon 362 in, for example, FIG. 14. When preop preparation information icon 362 has been, an information field 382 is presented.

Pre-op information field 382 provides an icon 384 to a document constituting pre-op preparation instructions, and tapping on link 384 results and presentation of pre-op instructions containing detailed information for the patient to prepare himself or herself for the surgical procedure. When icon 384 is tapped on, the presentation illustrated in FIG. 15 is replaced by a full-screen scrollable text presentation of instructions with or without illustrations. For example, information respecting office visit, insurance caregiver/family involvement, preparation required before and actions to be taken after surgery, restrictions, activity and the date of a postoperative visit, may be presented.

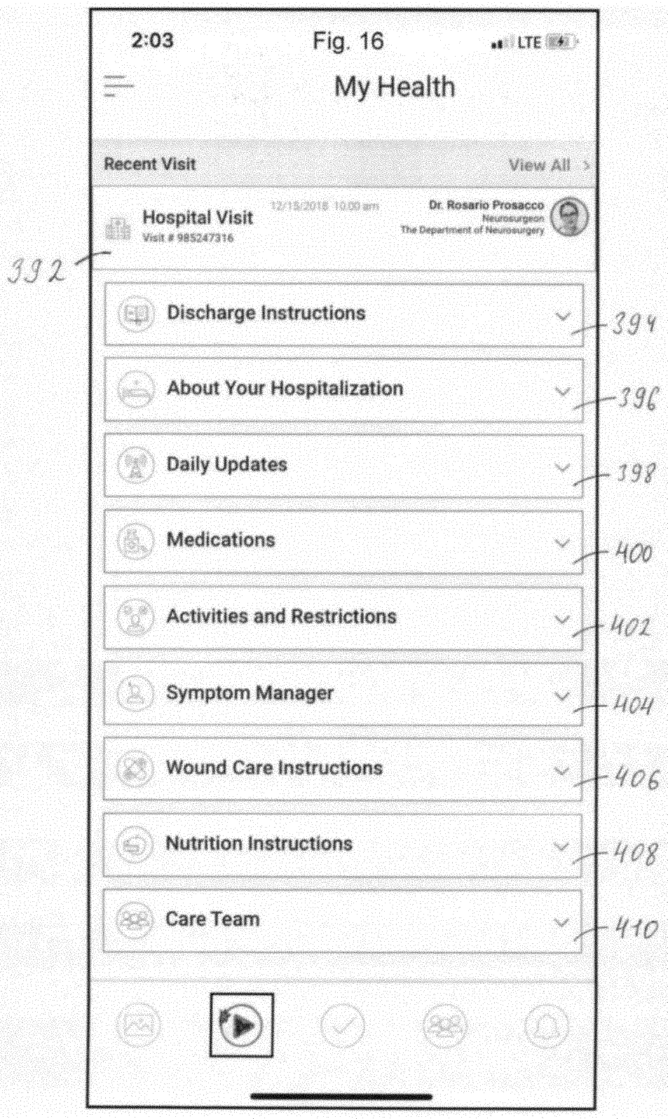

In the event that a patient has been scheduled for a surgery, typically the same is referred to in the inventive as a hospital visit. Information respecting such hospital visit is accessed by tapping on an icon associated with the surgeon performing the surgery, in the illustrated example Dr. Rosario Prosacco, a neurosurgeon. Generally, in accordance with the present invention, it is contemplated that information respecting a procedure, examination or other service may be accessed by clicking on the professional performing the service. Accordingly, clicking on icon 348 in FIG. 5 will bring up the informational screen illustrated in FIG. 16, which has information relating to a recent office visit with Dr. Rosario Prosacco. Recent caregiver's information such as phone number, location of work, email and so forth can be viewed by tapping on the photo on the right side of icon 392. Likewise, clicking on icon 390 in FIG. 27, which details the internal care team as discussed in detail below, will also bring up Dr. Rosario's information.

More critically, FIG. 16 presents icons linking to various items of information. Icons may optionally include the date or dates of a hospital visit(s) icon 392 (which also provides the visit number, name of the doctor and department), discharge instructions icon 394, hospitalization information icon 396, daily updates icon 398, medications icon 400, activities and restrictions icon 402, symptom manager icon 404, wound care instructions icon 406, nutrition instructions icon 408 and care team 410, as illustrated in FIG. 16.

Optionally, care team icon 410 instead of presenting complete care team information may only limit the presentation of information to those persons directly involved with the surgery.

In accordance with the invention, tapping on each of icons 392-410 brings up an associated respective screen for accessing information relating to the subject covered by each of the icons.

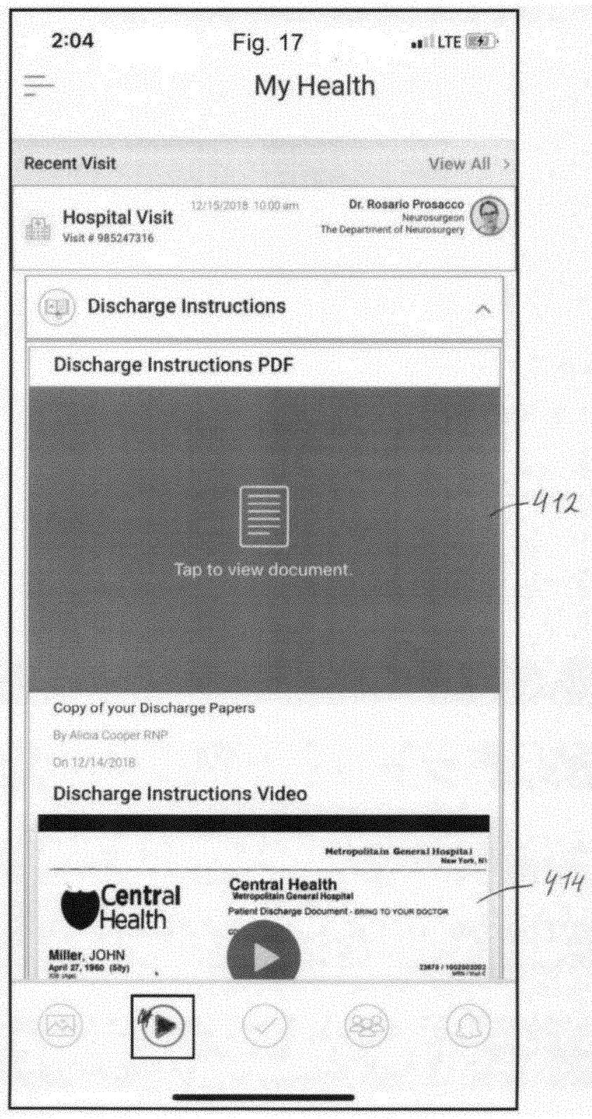

More particularly, discharge instructions comprising a text accessible by tapping on icon 412 and a video accessible by icon 414 may be accessed by tapping on icon 394 in FIG. 16, as illustrated in the screen illustrated in FIG. 17. This screen may also present links to a copy of discharge papers, other instructional video, location address, phone number, and case manager/social worker phone number and the like. If the information in hospital visit or any other category cannot be seen on a single screen, it can be viewed by scrolling up/down.

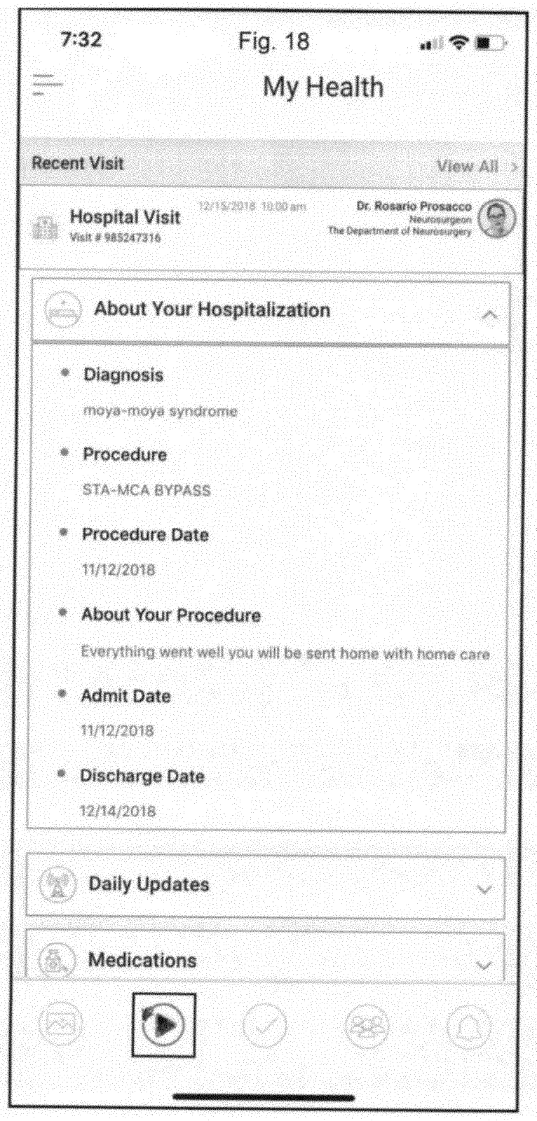

Hospitalization information may be accessed by clicking on icon 396, thus presenting the screen illustrated in illustrated in FIG. 18, which provides links to medical information and dates, diagnosis, procedure, procedure date, procedure information, admission date and discharge date.

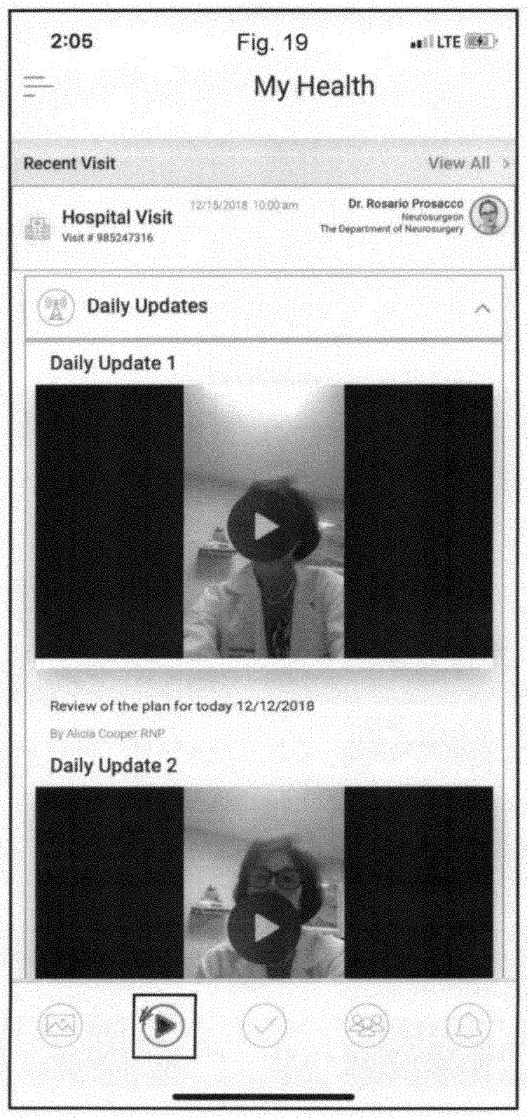

Daily updates may be accessed by clicking icon 398 in FIG. 16 through the presentation of a screen such as that illustrated in FIG. 19. More particularly, links may be presented to, for example, videos stored optionally in the chronological order and giving the name of the doctor and date. More particularly daily updates will review the plan for the same day, instructions, and give additional health feedback.

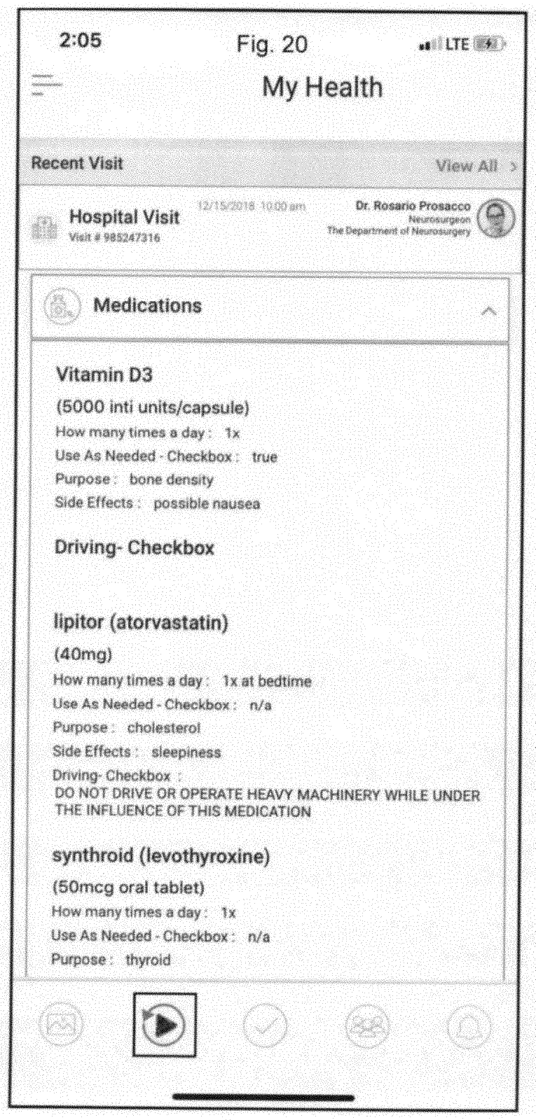

Medications icon 400 may be tapped on by the user patient to bring up the screen illustrated in FIG. 20. The screen contains detailed information related to medications and their use. More particularly, name of the medication, dosage, frequency of use, purpose and side effects. Optionally, in accordance with the invention, when the prescription is filled, the date on which it was filled may be indicated in the screen illustrated in FIG. 20. Alternatively, the presentation illustrated in FIG. 20 may indicate the date when the next refill needs to be made.

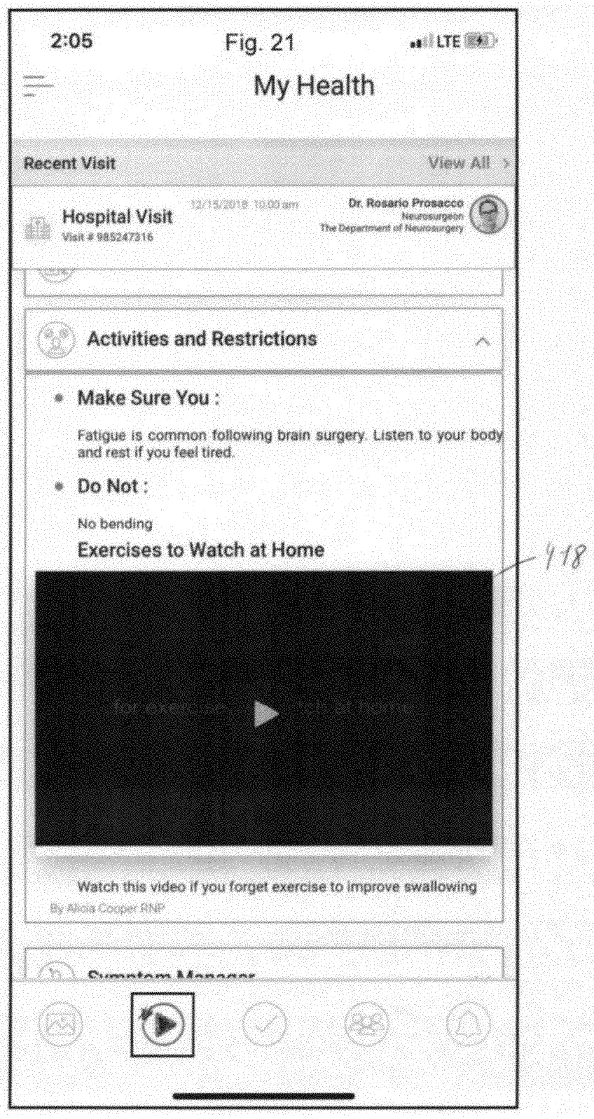

Tapping on Activities and Restrictions icon 402 will bring up the screen illustrated in FIG. 21, which is designed to inform the patient-user as toward allowed and/or recommended physical activities. Likewise, the screen may indicate exercises are necessary. In accordance with the invention, it is contemplated that a video describing and demonstrating each exercise may be accessed by one or more icons 418.

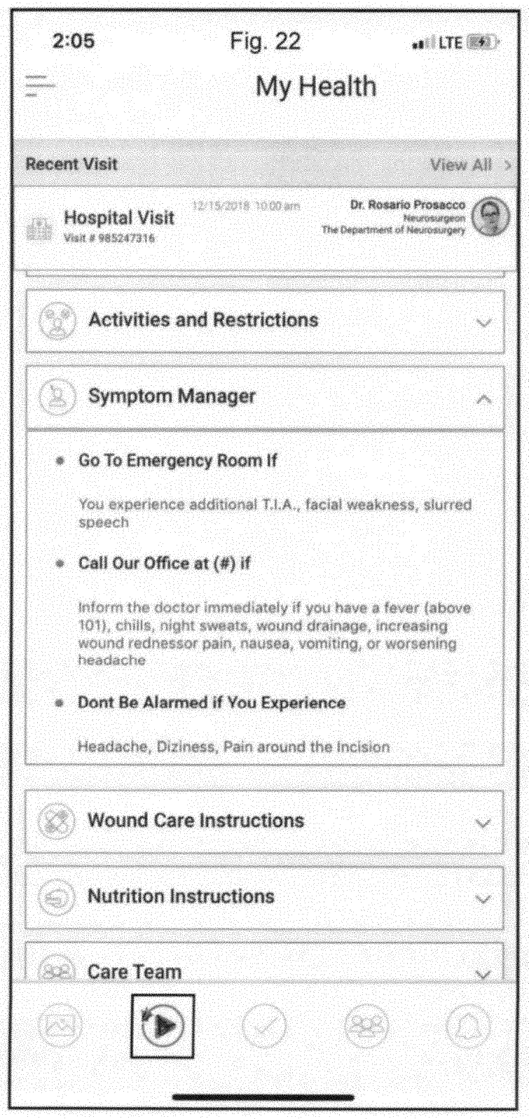

By tapping on Symptom Manager icon 404 in FIG. 16, the system presents the screen illustrated in FIG. 22, which contains information related to symptoms and how to take care of them. More particularly as illustrated in FIG. 22, the Symptom Manager screen identifies various symptoms in three categories. It also recommends appropriate particular actions to be taken by the patient in the event that particular symptoms are experienced. For example, some symptoms may require going to the emergency room, or making a call to the office of a care team member. Other symptoms require no action of the same is communicated by the system to the patient by a message to not be alarmed.

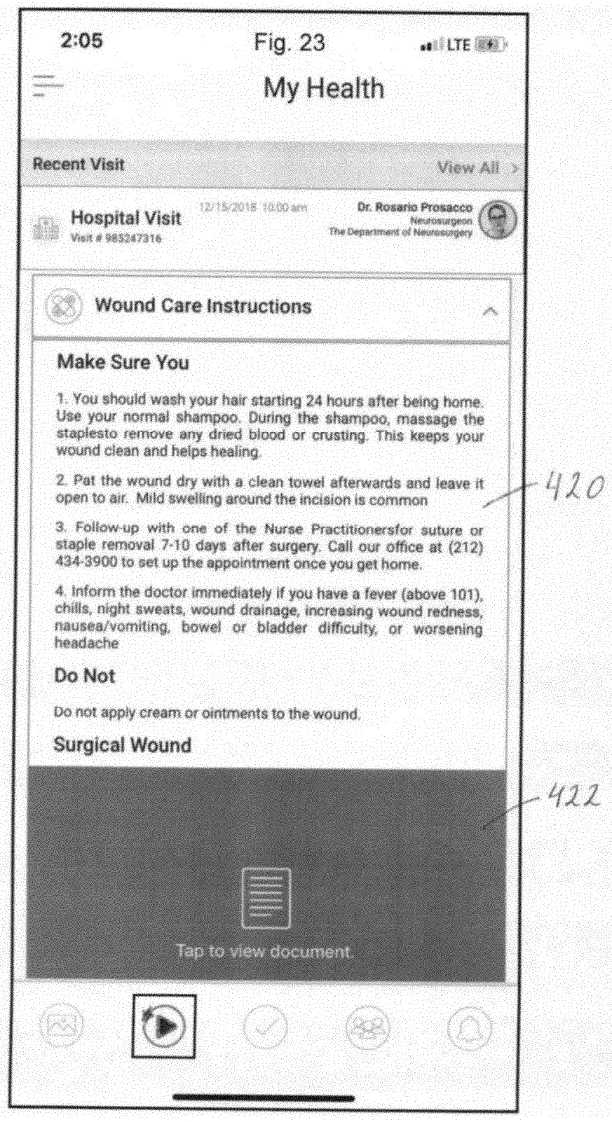

Tapping on Wound Care instructions icon 406 brings up detailed wound care directions on how to take care of a wound, for example a surgical wound, for example by bringing up the screen illustrated in FIG. 23. In accordance with the invention, a summary may be provided in a text field 420. In addition of full more detailed document may be brought up by tapping on icon 422. More particularly, this may comprise instructions on actions to be taken that will contribute to healing, such as washing, application of medications, and raising of a wounded area to relieve pressure. Likewise, necessary actions to be taken in the event that problems were particular symptoms arise. In addition, such instructions may include information on what not to do.

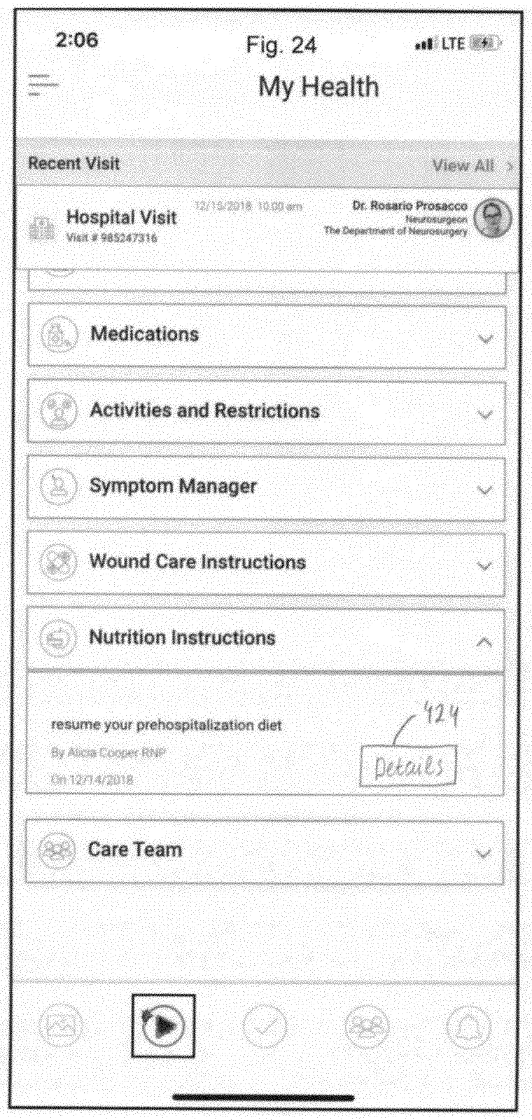

Tapping on Nutrition Instruction icon 408 in FIG. 16 will bring up the information access screen illustrated in FIG. 24, which contains detailed information about the diet that should be followed by patient, name of the doctor, and dates. Here again relatively short instructions may be provided in the screen of FIG. 24, while clicking on a “details” icon 424 will bring up a more complete document.

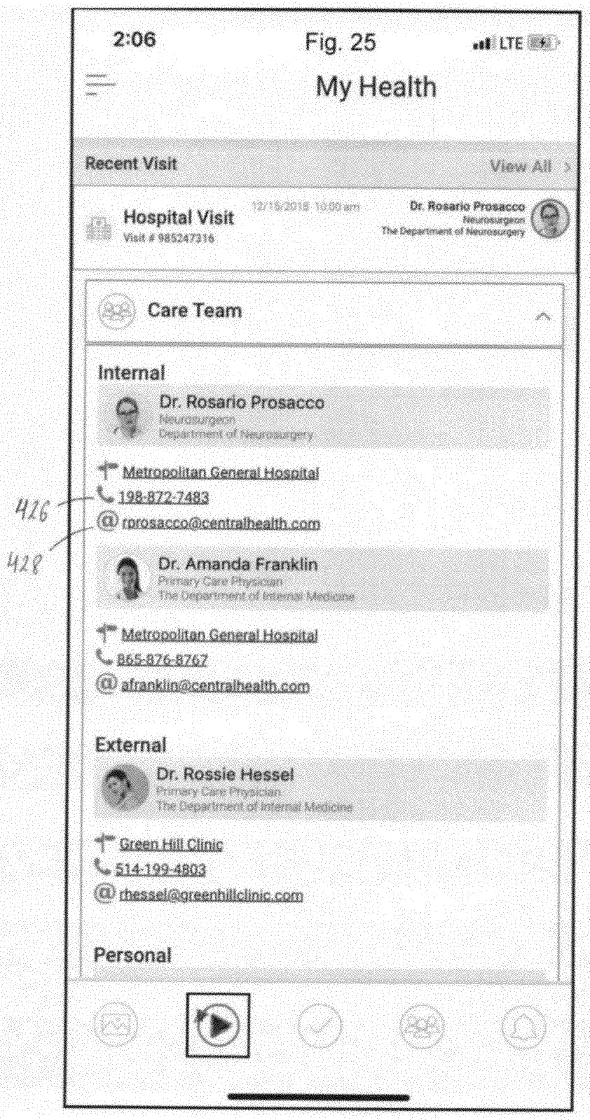

Tapping on Care Team icon 410 in FIG. 16 will bring up the screen illustrated in FIG. 25, which contains information related to care team, and is similar or identical to the option 130 illustrated in FIG. 25. Optionally, only members of the care team associated with the hospital visit, to which the screen of FIG. 16 is dedicated, may be presented in the screen of FIG. 25. In addition, contact information, including telephone numbers 426, email addresses 428 and professional identification information may also be presented.

In accordance with the invention, it is contemplated that the subject that would be loaded on a conventional smartphone. Accordingly, further in accordance with the invention, clicking on a telephone number 426 will result in placing a call to that number. Likewise, clicking on an email address 428 will bring up a blank email document addressed to the individual as that email address and in which an inquiry, request, concern or other communication may be entered by the patient user for sending to the care team member.

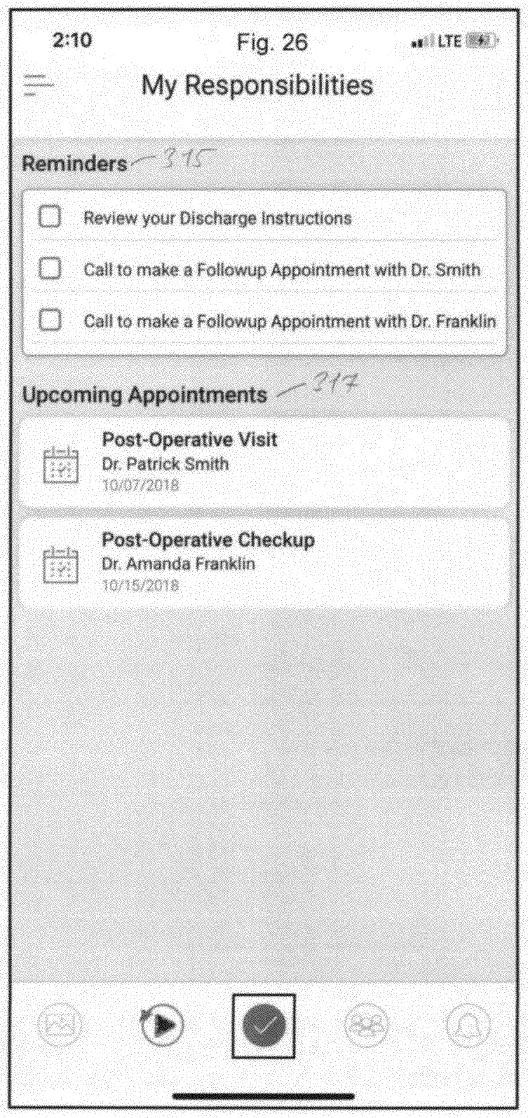

In accordance with the invention, tapping on the “My Responsibilities” icon 314 will bring up the screen of FIG. 26 which contains information related to the actions that should be taken by the patient. More particularly, reminders of steps that should be done and upcoming appointments with dates, name of doctor, place and purpose may be presented in accordance with the invention in the screen of FIG. 26. For example, reminders under icon 315 display instructions that are recommended to review or necessary appointments and upcoming appointments under icon 317 that display date, location, and doctor's name.

Alternatively, each of the tasks which the patient is responsible for may be associated in the database of the inventive system with a time. At the appointed time, the patient may be emailed with a reminder to perform the particular task, and given the opportunity to check the same as being done, or to check a presented box indicating that the same will be done shortly and requesting a reminder. When the reminder is sent, the patient is again given the opportunity to indicate that the task is performed. In addition, optionally, if the task is not indicated is done, a family member or member of the professional team may be given an email indicating that the task is not yet performed.

Tapping on Care Team icon in, for example, the screen of FIG. 5 brings up the display of FIG. 27 identifying internal care team members including doctors and a registered nurse practitioner in the illustrated example. The screen of FIG. 27 also provides access to all caregivers of the patient-user including caregivers in three categories, namely, an internal, external and personal. Clicking on a caregiver provides information about each respective caregiver. More particularly by default it displays icon 431 which identifies internal caregivers.

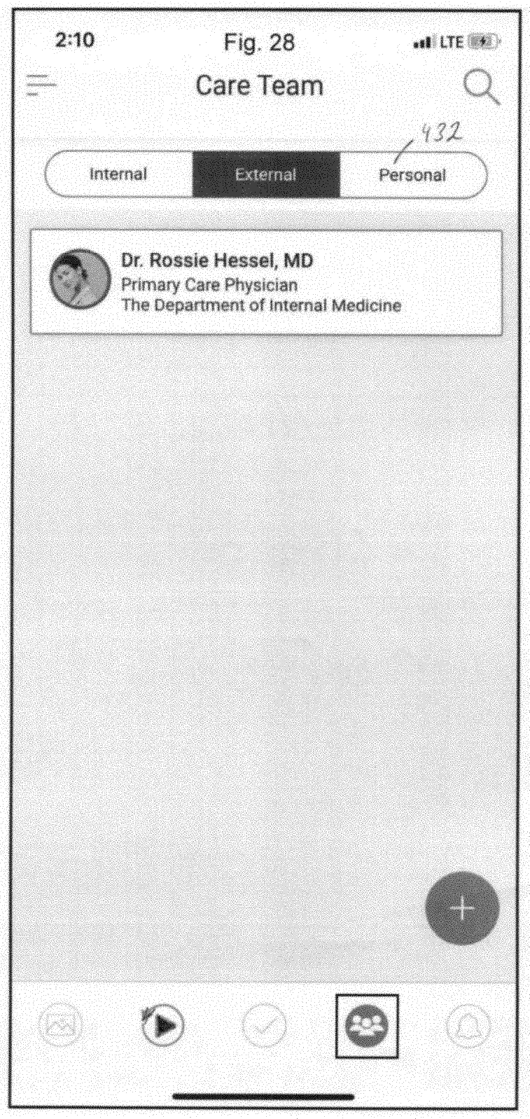

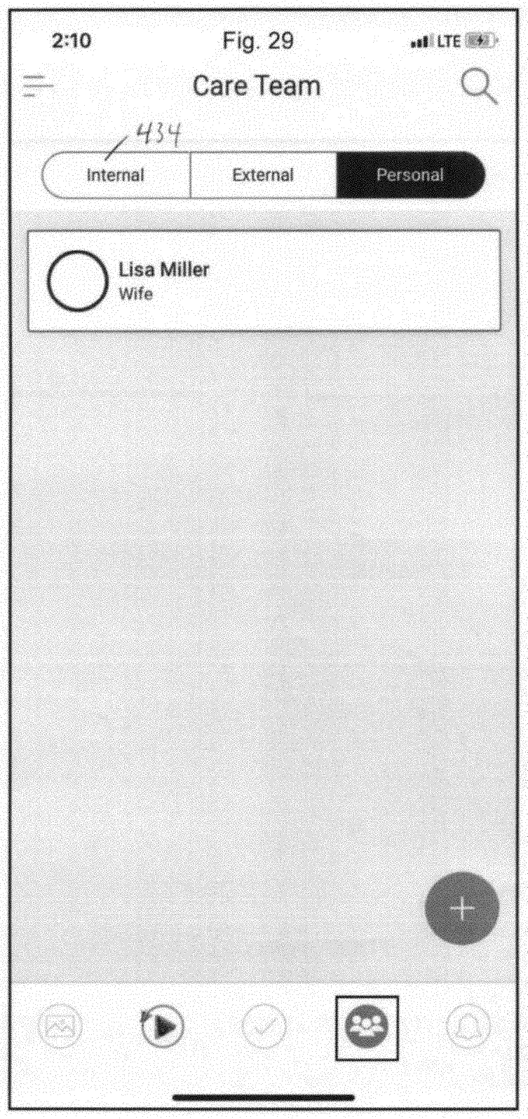

Clicking on icon 430 in the screen of FIG. 27 brings up the screen of FIG. 28 which identifies external caregivers. Similarly, clicking on icon 432 in FIG. 28 (or in any of the screens which includes icon 432) brings up the screen of FIG. 29 which lists personal caregivers, in the illustrated example, the wife of the user-patient.

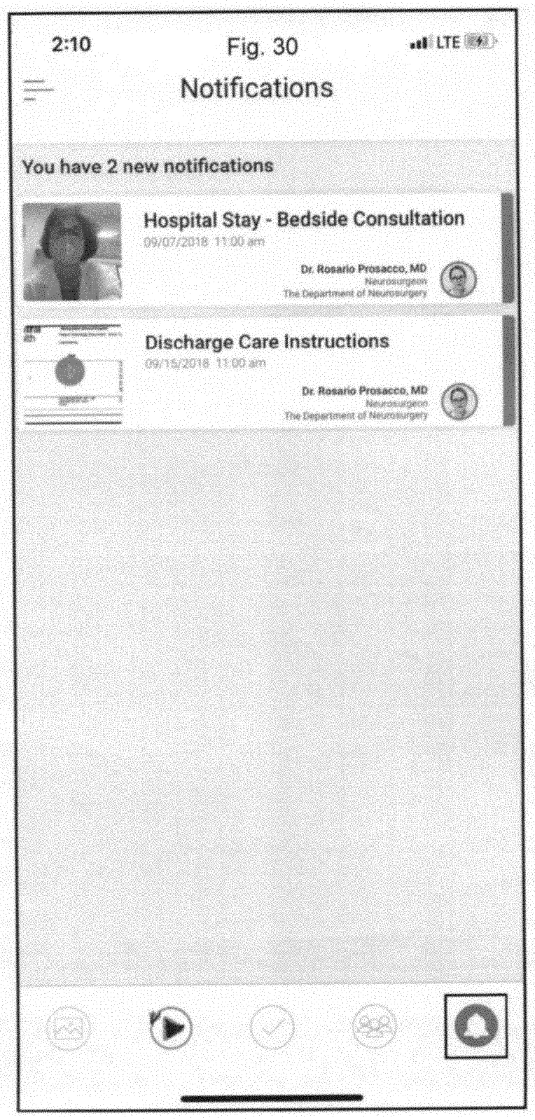

Tapping on Notifications icon 318 in any of the screens in which it appears brings up the display illustrated in FIG. 30. The screen presents information intended to notify the patient about updates, instructions, relatively urgent necessary actions, other actions, etc. It is contemplated that this information, like all the information in the system accessible by the various icons is supplemented and updated on a continuous basis as professionals using the system deem appropriate, and/or as certain actions are taken and automatically or manually recorded in the system, such as the fulfillment of prescriptions or the appearance for and performance of a surgical procedure.

In accordance with the invention, it is contemplated that the system will monitor the parameters which describe the use of the inventive system by the patient. For example, the system may look at the number of times that the patient uses certain features, for example, video instruction playback, textual information, communications features, and so forth, as described above. Using the frequency of use of particular features is anticipated to be useful in facilitating patient use of the system. For example if high-value features are not being utilized, the operator of the system may institute educational and instructional communications to guide the patient toward the same. Likewise, general patient parameters, such as satisfaction, success rate, complications, and so forth may also be identified using existing information in the system. As such information is gathered the same may be analyzed and used to design patient communications, position approaches, and the creation of information databases respecting success rates of various procedures, patient problems with various features, and so forth for presentation to medical care members, for example physicians and physicians assistants, surgeons, etc. In accordance with a particularly preferred embodiment of the invention, it is contemplated that patients will be enrolled into the inventive application without a password, at least in accordance with one embodiment of the invention. The reason for the same is the ability to use the identification of the patient device which is electronically immutable as a security device and a means to avoid use of the password. The password-less on boarding of patients onto the system initially, and the password-less accessibility of the system during the entire duration of the patients use, for example from initial examination through postoperative recuperation and rehabilitation periods, is believed of particular value in sofaras patients may be weak, distracted, in pain, and so forth, and relying on the identification of a known device as an alternative to requiring a password is believed to provide a net positive, more particularly a very strongly positive, advantage to both patients and the provider and treatment team. Further, in accordance with a particularly preferred embodiment of the invention, it is contemplated that a conventional QR code (or other code) patient identification may be integrated into the inventive apparatus. More particularly, during the normal course of treatment, the wristband of the patient is scanned. When that scan occurs in the normal course of treatment, the information that the patient is being treated is uploaded into the application for access by family, professional and other care team members. The location of the scanner, time of day and other parameters may be automatically input into the system to yield additional information.

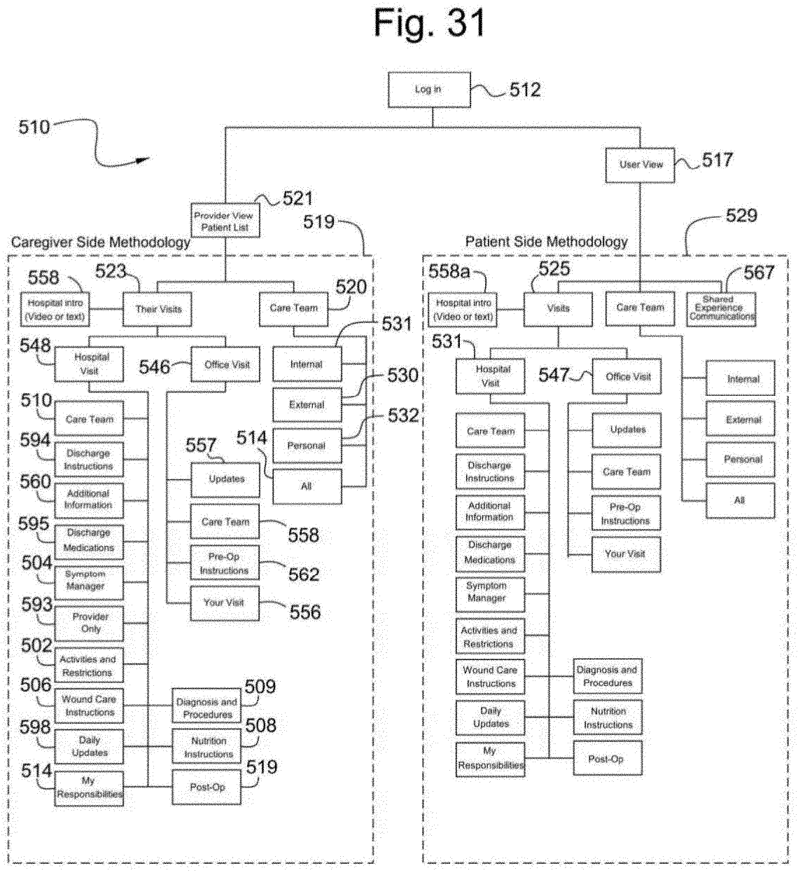

Referring to FIG. 31, an alternative method 510 similar to method 310 illustrated in FIG. 4 is shown. Generally, FIG. 31 illustrates an alternative methodology associated with the present invention in the context of a caregiver side (i.e. provider/doctor) methodology 519, which, for purposes of convenience, will be referred to as a caregiver app; and a patient side (i.e. user/patient) methodology 529, which, for purposes of convenience, will be referred to as a patient app. FIG. 31 represents the methodology of a scheme for both storing and accessing information.

More particularly, FIG. 31 represents an application of the inventive method to, for example, mobile devices, such as devices operating on the iOS 12 operating system or the android operating system. Method 510 constitutes an exemplary embodiment of the method of the present invention, which may be implemented on the IT infrastructure illustrated in FIG. 1.

In accordance with the present invention, inventive method 510 may be implemented on any suitable computing electronic infrastructure, such as one comprising a central server (used by the operator of the system of the present invention) and a plurality of smart phones (used by healthcare provider personnel, on the one hand and patients and family team members on the other.

Generally, it is noted that the methodology illustrated in FIG. 31 is implemented by software on individual doctor, patient, and other smart phones which in cooperation with software on server 26 enables the methodology illustrated in FIG. 31. More particularly, the smart phone may be little more than an interface for accessing functionality on server 26. Alternatively, doctor, patient and other user smart phones may have respective applications representing a robust software implementation providing a great portion of the functionality reflected in the methodology of method 510. The further alternative implementation may be used in which the performance of various functional features is more or less evenly divided between the patient or doctor computing device and the server.

Likewise, it is possible to implement the invention with different apps being loaded onto user smart phones, for example, a caregiver application, and patient application. It is also possible in accordance with the present invention to have still further different types of applications, such as a doctor application, a nurse application, a patient application, a technician application, a family member application, and so forth. However, as a practical matter it may be advantageous to have a single application downloaded by all users, and that when the user signs in at step 512 and gives the user's credentials, the software resident on server 26 will make accessible those functionalities appropriate to the particular user, whether that user be a doctor, radiation therapy technician, patient, patient family member, and so forth.

For purposes of organization, the alternative embodiment of the invention illustrated in FIG. 31 is, where practical, numbered with numerical part designators which are multiples of 100 different from the numbers assigned to corresponding, analogous or similar parts in other embodiments.

Referring to FIG. 31, in accordance with the present invention, the inventive method 510 may be initiated at step 512 by a provider/doctor or user/patient (or other user) who has installed an app on his/her smartphone or other suitable electronic computing device logging into the system at step 512. As alluded to above, while it is contemplated that most if not all users will access an interface with the inventive system via smart phones, the inventive system may also be made available to other types of computing devices, such as personal computers, netbooks, and so forth.

As alluded to above, providers such as doctors, nurses, technicians, and so forth will have a caregiver app 519 installed on their smart phones. Likewise, patients, family team members, friends patient application 529 installed on their smart phones. Applications 519 and 529 provide different functionalities customized to the needs of the two (or more) groups using these applications. The app is structured to implement, on an electronic computing device the method illustrated in FIG. 31 as is more fully explained below. More particularly, in FIG. 31, method steps allow access to information in “chapters” as indicated by the descriptive designations in FIG. 31, which are associated with touch activated hyperlinks in the application.

More particularly, method 510, is implemented through an electronic computing device, such as server 26 in FIG. 1. Server 26 thus implements the methodology which consists of two different parts, one of which (caregiver app 519) is associated with provider/doctor and another application 529 associated with the user/patient. In accordance with method 510, when the user logs in at step 512 and provides his/her credentials, if the user is a doctor, a provider view is provided at step 521 (which consists of a list of patients under the care of the particular provider) and server 26 is signaled to provide the methodology illustrated in caregiver “app” 519 (for example the caregiver side of a single app downloaded by all users). As noted above, the system determines which part to initiate based on the login information that is entered on step 512. That is, depending on the log in information entered at step 512, provider/doctor view 521 or user/patient view 517 will be displayed (or, alternatively, specialized views which may be provided to nurses, radiation technician operators, and/or others).

For the sake of clarity, it is noted that the provider/doctor may be an employee of a medical office or hospital and the user/patient is a person that needs medical attention. A group of people in the office or hospital that give medical attention to the user/patient are, in accordance with the invention, typically members of the user/patient's care team, together with the patient's internist, surgeon, anesthesiologist and perhaps others.

Similar to the methodology described above in connection with the embodiment of FIGS. 1-30, a provider/doctor may use his electronic computing device (for example smart phone or personal computer), which has had the app downloaded onto it, to access all information generated for, collected from and otherwise associated with the user/patient in accordance with the general methodology disclosed in connection with the description of, for example, FIGS. 1-3. When a provider/doctor logs in at step 512, the user/patient list will be displayed at step 521. The provider/doctor then may tap on a desired user/patient's icon (which may simply be the name of the patient with or without a thumbnail photograph) from the list displayed at step 521 and retrieve information associated with the specific patient.

Such retrieved information generally comprises information typically compiled into the patient's record to enable quality care for the particular patient. Also as alluded to, above, the inventive system may also have collections of facility specific information meant primarily for patients but also made accessible to medical professionals so that they are aware of information presented to patients. Such information may consist of a hospital introduction, for example a video, which can be presented to the doctor at step 558, where the doctor can opt to make it accessible to the patient. As discussed above, this may include video, audio, pictures, and text records of such things as patient and doctor interactions, physical examination of patients, doctors giving patients a diagnosis, doctor notes, and so forth.

Elements included within caregiver side methodology 519, which can be accessed by health professionals from the screen provided at step 521, are in many respects substantially similar to the elements of method 312 of FIG. 4.

Referring back to FIG. 31, after being presented with the provider screen at step 521, the provider/doctor, as noted above, selects the name of the patient and is provided with links to information on that patient. In accordance with the invention, it is contemplated that three hyperlinks will be presented. The first of these hyperlinks provides access to, for example, the video described above. The second hyperlink provides access to the visit history for the patient. When the second hyperlink is activated, it provides access to the visit history of the patient, for example by displaying a list of visits (for example listed by date), information about which may be accessed by clicking on the particular visit, and this information may be presented on the screen of the smart phone at step 523. The screen presented at step 523 may provide information on the various visits of the patient in a separate screen at step 523. Alternatively, accessible information on the smart phone may be limited at step 523, for example to the last three visits, with options being provided on the screen to access earlier visits. The object is to simplify the presentation of information on the screen.

A third hyperlink, when it is clicked on, provides access to information on professionals assigned to the patient which is accessed at step 520. The link also enables editing of the information stored in that chapter by providing hyperlinks which trigger steps 514, 530, 531 and/or 532. That information may be accessed by presenting at step 520 a caregiver list, perhaps associated with the caregiver specialty, such as surgeon, internist, anesthesiologist, etc. Each of the names on the list may act as a hyperlink. When clicked on, the hyperlink associated with a particular caregiver results in a display of various information for the caregiver, such as his contact information, location, and so forth. Alternatively, the list may be associated with, for example, the status of the caregiver as personal, hospital internal professional, and hospital external professional.

In accordance with a preferred embodiment of the invention, at step 520, instead of a simple list of all caregivers, the display may comprise four hyperlinks which, when clicked upon, present different parts of the care team. For example, the four hyperlinks may be specific to caregivers internal to the hospital (such as a surgeon), caregivers located outside the hospital such as cancer radiation therapy providers, personal caregivers, such as in home caregivers, and finally a fourth icon may provide access to all caregivers in a single list. These lists are presented for simple display and/or editing at steps 514, 530, 531 and 532. After navigating to hyperlinks presented at step 520′, the system presents to patients information on caregivers at steps editing at steps 514′, 530′, 531′ and 532′. As alluded to above, the screen presented at step 521 has in addition to the care team hyperlink with the functionality described above, a hyperlink which when activated results, in accordance with a particularly preferred embodiment of the invention, in the presentation of a pair of hyperlinks connecting to information at steps 546 and 548 corresponding to office visits and hospital visits.

If hyperlink at step 546 is clicked on, the screen is presented with hyperlinks leading to presentations of information for the particular patient (such as, updated medication information, the date of the next visit with the name of the professional being visited, changes in caretaker contact information, latest information on tests, visit diagnoses and the like, payment information, procedure cost and insurance, insurance status, and other information as may be directed by the operator of the system and/or professionals responsible for patient care), care team information for the particular patient (names, contact information, specialties, care team member qualifications, location of the patient and so forth, messages such as emails or texts from patients and meant for the particular care team member, patient ratings for care team members, patient complaints, patient concerns, patient questions, and so forth), pre-operative instructions for the particular patient, and a display of visit information (such as test results, diagnoses, date of next visit, new prescriptions given to the patient during the particular visit, new diagnoses during the particular visit and or other items).

Office visit information which may be entered by the professional users at step 546, can be accessed by patients at step 547, after navigating through step 525. Office visit information input by professional caregivers after navigation by way of step 546 may include the following exemplary chapters giving information on the particular subject matter of the chapter. More particularly, at step 546 a menu of hyperlinks corresponding to steps 557, 558, 562 and 556 may be presented with alphanumeric markings corresponding to their content for the purpose of implementing professional input into these chapters. For example, information respecting a patient's visit may be input at step 556 upon the clicking of the appropriate hyperlink. Likewise, by clicking (for example by touching) the Care Team icon at step 558 information on the care team may be input by professionals. Likewise, professionals may input Pre-Op Instructions at step 562, in a manner similar to Office Visit 346 (FIG. 4). On the patient side, office visit information may be accessed by corresponding descriptively labeled hyperlinks presented at step 547. Such information may include updates presented at step 557′ which enables display of updates done by provider/doctor, and organized according to the chapters of office visit information chapters navigated to by way of step 546, optionally sorted by date. Likewise, by accessing the appropriate hyperlinks the system presents at step 558′ care team information, at step 562′ pre-op instructions, and at step 556′ information respecting the patient's visit. It is noted that on the caregiver side of the method the methodology diagram may be used to input and retrieve information. Limited opportunities for input of information may also be presented on the patient side methodology of the present invention. Detailed care team information may also be accessed at step 520′.

If the patient selects hospital visit information at step 548′ after navigating there via step 525, and clicks on hyperlinks presented at step 548′ and corresponding to steps 510′, 594′, 560′, 595′, 504′, 502′, 506′, 598′, 514′, 509′, 508′ and 519′, the system acts to provide information corresponding to a plurality of options, for example information corresponding to chapters whose content was input by doctors or other health care professionals at corresponding and similarly numbered steps 510, 594, 560, 595, 504, 502, 506, 598, 514, 509, 508 and 519, also as illustrated in FIG. 31. The information at each of these hyperlinks are referred to as chapters of information available in the system.

Hospital visit information which may be selected at step 548 may include exemplary chapters of information which may be accessed by system users, some of which are similar to exemplary chapters of hospital visit at step 348 (FIG. 4) as is apparent from the substantially similar names of the various chapters in FIG. 31 compared to FIG. 4. However, FIG. 31 includes additional chapters of information which may be accessed at a plurality of steps, including a discharge medications information input step 595, which may be presented in the form of a hyperlink to a discharge medications informational chapter in the databases of the inventive system. When the same is clicked on, a listing of the medications prescribed for the patient, for example upon discharge after a surgical procedure, is presented together with dosage size, frequency of administration, potential side effects and danger signs, name of the dispensing pharmacy, and other associated information as may be deemed appropriate by the prescribing physician. Discharge medications which the patient should be taking post-discharge are determined by reviewing: medications the patient was taking prior to admission, current medications (taken within previous 24-hour period), and new post-discharge medications. The same may be stored, remotely at the server of the system operator, for access by the inventive app at step 595. In addition, certain information only available to members of the professional team may be provided and edited upon clicking on a “Provider Only” hyperlink at step 593. Such “Provider Only” information may comprise messages created by care team providers and meant to be seen only by care team providers, hospital to hospital communication (including communication by nonprofessionals such as financial administrators, insurance administrators, and other such individuals). A hyperlink presented at step 509 may provide access to such things as diagnoses made by doctors, recommended or optional procedures, procedures which have been performed together with associated information, and so forth).

The system also presents a display of a post-op information via a chapter hyperlink 519. When hyperlink 519 is clicked on, the system presents a screen showing such things as post operation medications, post-operation cautions respecting physical activity, post operation cautions respecting diet, recommended diet, recommended resting positions or other physical cautions, possible indicators of problematic indicators and, if appropriate, instructions to contact a particular individual, and other information, if any deemed appropriate by the physician in charge or other healthcare professionals on the professional medical caregiver team. A hyperlink 560 may be used to present additional information.

The data related to user/patient's diagnosis and procedures is stored in the chapter input at step 509. This may contain information such as user/patient test results and diagnosis, examination results and diagnosis based on the results produced by the user/doctor's care team. Similarly, information related to post-surgery care may be stored by and made available to professionals (depending upon the privileges) at step 519. Post-surgery information may be, for example, a summary of possible post-surgery symptoms such as pain, itching, or discomfort.

When a patient or other nonprofessional user logs into the system at step 512, the patient is provided with a user view at step 517. The user view presents a hyperlink at step 558a which when clicked on results in the display of hospital information, such as a video or text information, substantially as described above in connection with the doctor/professional caretaker side of the application. From the screen on the patient side methodology, the patient can access all the information indicated in patient side methodology steps 529. It is noted that the information in patient side methodology steps 529 are substantially identical to those made available to doctors in the caregiver side methodology steps 519, as is indicated by the substantially identical chapters in patient side methodology steps 529. It is noted that patient side methodology steps 529 include chapters divided between hospital and office visits, and four chapters under care team. This compares with the contents of the caregiver side methodology steps 519 which comprises additional chapter information divided between hospital and office visits, and four chapters under care team. The additional chapter under the hospital visit category is “Provider Only” which is not made available to the patient.

At care team step 520, a professional may retrieve and/or edit information related to all care givers of the specific user/patient similarly to care team 320 of FIG. 4. Referring back to FIG. 31, in addition, care team access/edit step 520 provides access at step 521 to the list of all care givers such as internal, external and personal can be seen.

Generally, it is noted that much of the information provided on the professional caregiver/doctor side of the inventive methodology described above is identical to information provided to the patient, as will be described below. It is the object of the present invention to provide doctors and other professionals with this information so that they know what information is being presented to the patient. Armed with this information, doctors are able to give needed additional information to the patient in person, or, more importantly, to add additional information when required to the information available to the patient.

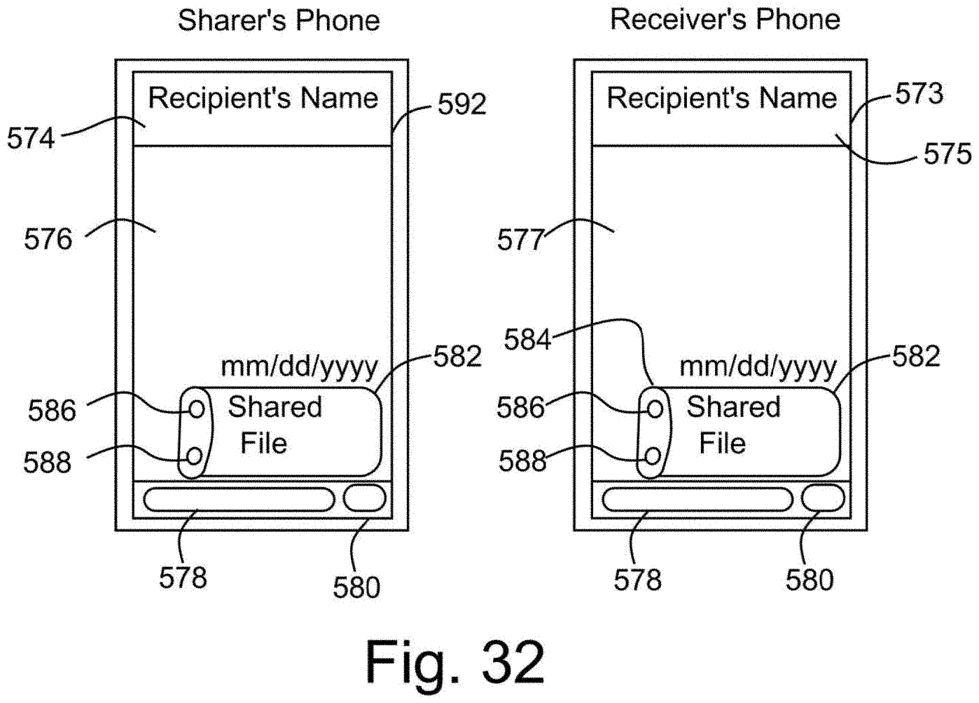

In addition, it is contemplated that the caregiver/doctor may use the inventive mobile app to create/upload/edit content such as documents, videos, pictures and so forth. Created content then may be shared with patient(s) and also with other caregiver(s)/doctor(s) via the inventive mobile app as is illustrated in FIG. 32, which corresponds to the screen on a person who is transmitting a file. The sharer of the content, for example a care professional, may select an item of content, performing appropriate gesture on said item to bring up a share menu option, and designate a member from a care team list presented at step 520, that will receive the shared information, and be presented with screen 572. The recipient's name will be displayed in box 574 and shared file names being shared between users are displayed in box 576. Shared files may be indicated as sent when they are sent. It is contemplated that, the inventive mobile app may thus be used as a resource that provides communication between patients and providers/doctors, without having to exit into a separate program.

The user may tap on box 578 to enter a text message or upload a file to send to recipient from care team list presented at step 520. Once the message or file is ready to be sent, the sharer sending the item tops on icon 580. Shared information 582 is then displayed in box 576 with the file identification 584. As shown on the face of the recipient's (i.e. receiver's) smart phone, the recipient is presented with a screen 573, on which he can see the sharer's name in a box 575. In box 577, recipient will see a shared file (optionally multiple files) and in particular, shared file 582 being marked as a received file with the date received.

When content is shared between inventive app users, the sharer and recipient of the content can see if the shared content has been opened. More particularly, icon 584 has a colored top, for example red, button 586 (indicating the content has not been read by the recipient) and a bottom button 588, color, for example in green, indicating that a file has been opened by the recipient. Once recipient opens the shared content 582 by tapping on it, red button 586 turns off and green button 588 turns on. This allows sharer to make sure that recipient has seen the shared content.

The inventive approach also prevents the redundant or conflicting presentation of information to the patient. In other words, all information given to the patient is presented by the system and may be viewed by all professional caretakers, if a caretaker is concerned or has a question about that information, the system may also provide the option of indicating the source of that information allowing the caretaker to contact the source of that particular information and resolve any questions, make suggestions, or participate in a group decision.

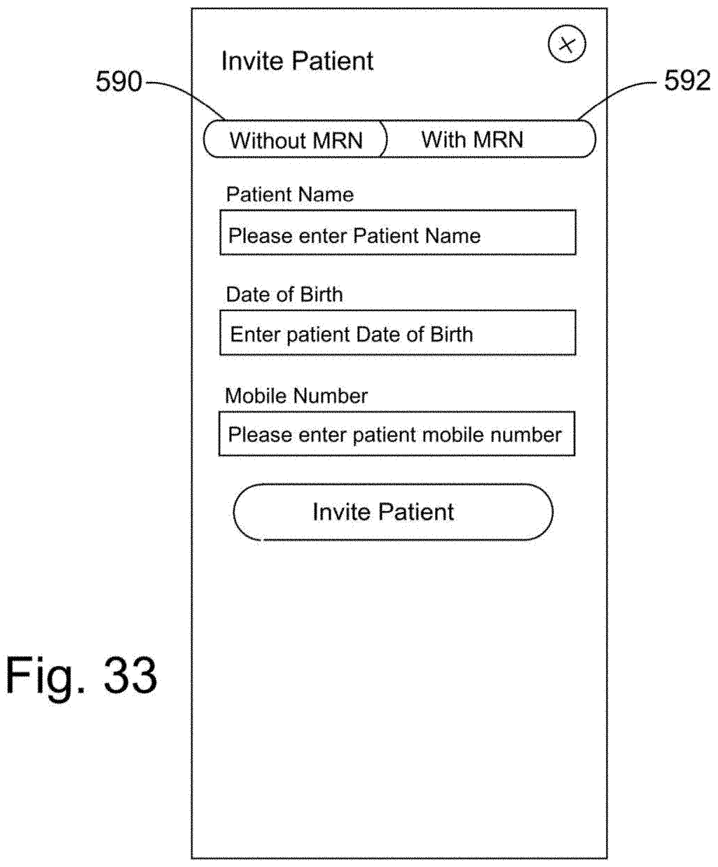

In accordance with the invention, any patient can download the application. However, in order to use the app in connection with their health care, patient needs to be invited out the system, for example by a doctor. Optionally, the provider/doctor may invite the person at step 521 (FIG. 31). The screen presented at step 521 an add patient icon. Once provider/doctor taps on add patient icon, a window that allows the doctor to invite a new patient appears as illustrated in FIG. 33. If the patient has a medical record number (“MRN”), the provider/doctor may tap on box 592 to use the MRN to invite the patient. For patients without an MRN, the doctor may invite the patient by filling in the patient name and other credentials by filling in appropriate fields as illustrated. If the patient accepts the invitation, the patient then has the electronic credentials to access inventive system.

The above described functionalities associated with the professional caregiver side methodology 519 are to a limited extent replicated in patient side methodology 529.

Given the coronavirus pandemic of 2020, it has been recognized that the inventive application will be of particular value in minimizing contact between doctors and patients, as well as different patients visiting a medical facility. At the same time the inventive system provides high-quality communications thus resulting in improved patient outcomes. The inventive communications infrastructure may have integrated therein videoconference and/or video chat capabilities to allow for family member contact with, for example, pandemic victims who are highly contagious, and also within the context of the care team and professional team assigned to the patient. Where the video communication is with a professional, the system may automatically track time and use artificial intelligence to determine whether the same is a billable event, or to gather, for example, time information to allow a human to determine whether such billing should occur. Optionally, recordings of video telehealth visits (and optionally family visits) may be made and maintained for a fixed period of time or permanently, and be made available to patients as a reference tool. It is further contemplated that whether or not such recordings are maintained permanently, patients will only have access to healthcare professional visit video recordings for a limited period of time in order to be certain that outdated information is not communicated. Likewise, as an alternative to video conferencing/video chat, the system may accommodate simple telephone communication within such structure.

While illustrative embodiments of the invention have been described, it is noted that various modifications will be apparent to and understood by those of ordinary skill in the art in view of the above description and drawings. More particularly, it is contemplated that system illustrated in FIG. 1 would serve multiple patients located at multiple hospitals, clinics and other health facilities. Such modifications are within the scope of the invention which is limited and defined only by the following claims.

1. A health care information generation and communication system, comprising:
(a) a body part image generation device for generating body part image information representing a body part of a patient;
(b) a body part image database coupled to receive the output of said body part image generation device and store said image information as a stored image;
(c) a stored image playback device coupled to said body part image database and generating a recovered image from said image information;
(d) a microphone;
(e) an image control device coupled to said stored image playback device to select a desired portion of said body part image information and output the selected portion as a selected image;
(f) a video generation device coupled to said image control device to receive the selected image from said stored image playback device and coupled to said microphone and combine the same into an output video, said output video comprising visual and audible elements;
(g) a video database coupled to receive the visual and audible elements of said output video from the output of said video generation device and store said visual and audible elements; and
(h) a video player for presenting a display of at least a portion of said visible and audible elements.

2. Apparatus as in claim 1, wherein said body part image information may be displayed as i) a plurality of two dimensional images representing different body parts, ii) views with different magnifications of one or more body parts, iii) different views of one or more body parts, or iv) partial views of one or more body parts.

3. Apparatus as in claim 1, wherein said body part image information is selected from the group consisting of i) still images, ii) moving images, iii) x-ray images, iv) ultrasound images, v) optical images, vi) mri images, and vii) other medical images.

4. Apparatus as in claim 1, wherein said recovered image is a two-dimensional image.

5. Apparatus as in claim 1, further comprising:
(i) an input device selected from the group consisting of a tablet, a touchscreen and an alpha numeric generating device.

6. Apparatus as in claim 1, further comprising:
(i) a video display device for displaying said output video as it is being generated in real time;
(j) touchscreen elements associated with said video display device or a tablet, said touchscreen elements or tablet being configured to receive a manual input, such as a circle encircling a part of an image displayed on said video display device from a person operating said video generation device; and
(k) an alpha numeric generating device coupled to input alphanumeric information into said video generation device to implement display of said alphanumeric information in said output video.

7. Apparatus as in claim 6, wherein said video generation device comprises non-volatile storage medium having stored thereon a template for said output video, said template presenting directions to said person operating said video generation device and presenting screens for the entry of alphanumeric information to be incorporated into said output video.

8. Apparatus as in claim 7, further comprising:
(l) alphanumeric data generating healthcare instrumentation generating alphanumeric data, said alphanumeric data generating healthcare instrumentation being coupled to said video generation device, said video generation device being responsive to a control signal input by a person operating said video generation device to incorporate at least a portion of said alphanumeric data into said output video.

9. A health care information generation, storage and communication system, comprising:
(a) a body part image generation device for generating body part image information representing a body part of a patient, and identification information for associating said body part image information with a particular patient;
(b) a body part image database coupled to receive the body part image information and its respective identification information, and store said image information as a stored image associated with its respective identification information;
(c) a stored image playback device coupled to said body part image database and generating a recovered image from said image information;
(d) a microphone;
(e) an image control device coupled to said stored image playback device to select a desired portion of said body part image information associated with a particular patient and output the selected portion as a selected image associated with the particular patient;
(f) a video generation device coupled to said image control device to receive the selected image from said stored image playback device and coupled to said microphone and combine the same into an output video, said output video comprising visual and audible elements, and said video being associated with said particular individual patient;
(g) a video and patient record database divided into a plurality of patient sectors, each of said patient sectors associated with an individual patient, said video database coupled to receive the visual and audible elements of said output video from the output of said video generation device and store said visual and audible elements in a patient sector associated with said particular individual patient;
(h) a publically accessible network;
(i) a server for making information in said video database available over said publically accessible network; and
(j) a patient smartphone associated with said particular individual patient, and having a smartphone memory and storing an application recorded in its smartphone memory for providing patient specific identification information and accessing said server over said publically accessible network to cause said server to access said video database and transmit said video associated with said particular individual patient to said patient smartphone.

10. Apparatus as in claim 9, further comprising:
(i) an input device selected from the group consisting of a tablet, a touchscreen and an alpha numeric generating device.

11. Apparatus as in claim 10, further comprising:
(i) a video display device for displaying said output video as it is being generated in real time;
(j) touchscreen elements associated with said video display device or a tablet, said touchscreen elements or tablet being configured to receive a manual input, such as a circle encircling a part of an image displayed on said video display device from a person operating said video generation device; and
(k) an alpha numeric generating device coupled to input alphanumeric information into said video generation device to implement display of said alphanumeric information in said output video.

12. Apparatus as in claim 11, wherein said video generation device comprises non-volatile storage medium having stored thereon a template for said output video, said template presenting directions to said person operating said video generation device and presenting screens for the entry of alphanumeric information to be incorporated into said output video.

13. Apparatus as in claim 12, further comprising:
(l) alphanumeric data generating healthcare instrumentation generating alphanumeric data, said alphanumeric data generating healthcare instrumentation being coupled to said video generation device, said video generation device being responsive to a control signal input by a person operating said video generation device to incorporate at least a portion of said alphanumeric data into said output video.

14. Apparatus as in claim 11, further comprising programming instructions presenting screens to the patient for enabling the patient to access a healthcare provider or other person associated with the medical treatment of the patient by way of email and/or telephone.

15. A method for a healthcare provider to communicate information to a person being treated, comprising:
(a) creating an image of a treatment protocol; prescription, drug taking directions, exercise directions
(b) creating an image of apart of the body related to a physiological issue; lung, ear pressure, x ray, mri
(c) inputting a still and/or video image into a video recording system while creating an audiovisual sequence; multiple
(d) inputting audio signal, said audio signal being generated from the voice of a healthcare provider, into said video recording system while said inputting a still and/or video image into a video recording system is in progress, to incorporate said audio signal into said audiovisual sequence; and
(e) making said audiovisual sequence available over a network accessible to said patient.

16. A method as in claim 15, wherein a patient record, said patient record comprising background information on the patient, such as medications, allergies, symptoms, medical history and the like, said patient record being created in or about the time of admission of the patient and associated with a particular individual patient, and said patient record stored on a video and patient record database divided into a plurality of patient sectors, each of said patient sectors associated with an individual patient, said video database coupled to receive said patient record in a patient sector associated with said particular individual patient.

17. A method as in claim 15, wherein said inputting of said still and/or video image and said audio signal is done in conjunction with manual markups of images on the screen of a video creation device.

18. A method as in claim 17, wherein said inputting of said still and/or video image and said audio signal is performed during the time that the patient is listening to and/or discussing his condition with his doctor.

19. A method as in claim 16, wherein the patient record includes each of a plurality of tasks which the patient is responsible for and appointed times to reach, and further comprising, at the appointed time, the patient is emailed with a reminder to perform the particular task, and given the opportunity to confirmation the same as being done, upon the failure to receive such a confirmation, a family member or member of the professional team is notified that the task is not yet performed.

20. A method as in claim 16, wherein the patient record is archived in a form which may not be altered in order to serve as a permanent record to guide future actions.

21. A method as in claim 14, wherein a manual input is incorporated into the audiovisual sequence to add manually generated image elements to the audiovisual sequence.
